(19) United States
(12) Patent Application Publication
(10) Pub. No.: US 2024/0317691 A1
(43) Pub. Date: Sep. 26, 2024

(54) Compounds for Modulating Epithelial 15-(S)-Lipoxygenase-2 and Methods of Use for Same

(71) Applicant: The Regents of the University of California, Oakland, CA (US)

(72) Inventors: Theodore R. Holman, Santa Cruz, CA (US); Wan-Chen Tsai, Santa Cruz, CA (US); Matthew Jacobson, San Francisco, CA (US)

(21) Appl. No.: 18/578,617

(22) PCT Filed: Jul. 18, 2022
(86) PCT No.: PCT/US2022/037497

§ 371 Date: Jan. 11, 2024

Related U.S. Application Data
(60) Provisional application No. 63/227,814, filed on Jul. 30, 2021.

(51) Int. Cl. C07D 239/00; A61K 31/4164

(52) U.S. Cl. CPC C07D 239/00; A61K 31/4164

(57) ABSTRACT

Compounds for inhibiting human epithelial 15-lipoxygenase-2 (h15-LOX-2) are provided. Compounds according to certain embodiments modulate ferroptosis and generation of hydroperoxy eicosatetraenoic acids (HpETEs). In some embodiments, compounds described herein modulate eicosanoid mediator biosynthesis from leukotrienes (LTs) to pro-resolving mediator class of lipoxins (LXs). Methods for treating or preventing a human epithelial 15-lipoxygenase-2(h15-LOX-2)-mediated disease are also provided. Compositions for practicing the subject methods are also described.

### CROSS REFERENCE TO RELATED APPLICATIONS

Pursuant to 35 U.S.C. § 119 (e), this application claims priority to the filing date of U.S. Provisional Patent Application Ser. No. 63/227,814 filed Jul. 30, 2021; the disclosure of which application is incorporated herein by reference in its entirety.

### STATEMENT OF GOVERNMENT SUPPORT

This invention was made with Government support under Grant No. AG058165 awarded by the National Institutes of Health. The Government has certain rights in the invention.

### INTRODUCTION

Lipoxygenases catalyze the peroxidation of fatty acids which contain bisallylic hydrogens between two cis double bonds, such as in linoleic acid (LA) and arachidonic acid (AA). Lipoxygenases are named according to their product specificity with AA as the substrate because AA is the precursor of many active lipid metabolites that are involved in a number of significant disease states. The human genome contains six functional human lipoxygenases (LOX) genes (ALOX5, ALOX12, ALOX12B, ALOX15, ALOX15B, eLOX3) encoding for six different human LOX isoforms (h5-LOX, h12S-LOX, hl2R-LOX, hl5-LOX-1, h15-LOX-2, eLOX3, respectively). The biological role in health and disease for each LOX isozyme varies dramatically, ranging from asthma to diabetes or stroke.

Human epithelial 15-(S)-lipoxygenase-2 (h15-LOX-2) is expressed in in macrophages, neutrophils, skin, hair roots, and prostate. h15-LOX-2 is also expressed in atherosclerotic plaques and linked to the progression of macrophages to foam cells, which are present in atherosclerotic plaques. Silencing the ALOX15B gene in human macrophages leads to decreased cellular lipid accumulation, a major factor in foam cell formation and plaque accumulation. h15-LOX-2 mRNA levels are highly elevated in human macrophages isolated from carotid atherosclerotic lesions of symptomatic patients. Additionally, h15-LOX-2 has been shown to play a central role in the “class switch” of eicosanoid mediator biosynthesis from leukotrienes (LTs) to the anti-inflammatory and specialized pro-resolving mediator class of lipoxins (LXs) in the airways. Reduced expression level of h15-LOX-2 in the lower airways was associated with a depressed LXA4/LTB4 ratio which contributed to cystic fibrosis (CF) lung disease. The h15-LOX-2/PEBP1 complex is also a regulator of ferroptosis, with PEBP1 acting as a rheostat by changing h15-LOX-2 substrate specificity from free PUFA to PUFA-PE, leading to the generation of HpETE-PEs.

### SUMMARY

Compounds for inhibiting human epithelial 15-lipoxygenase-2 (h15-LOX-2) are provided. Compounds according to certain embodiments modulate ferroptosis and generation of hydroperoxy eicosatetraeneoic acids (HpETEs). In some embodiments, compounds described herein modulate eicosanoid mediator biosynthesis from leukotrienes (LTs) to pro-resolving mediator class of lipoxins (LXs). Methods for treating or preventing a human epithelial 15-lipoxygenase-2 (h15-LOX-2)-mediated disease are also provided. Compositions for practicing the subject methods are also described.

In some embodiments, compounds of interest include a compound of formula (I):

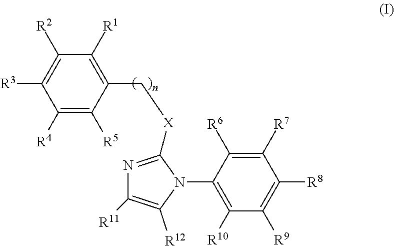

wherein R¹, R², R³, R⁴, R⁵, R⁶, R⁷, R⁸, R⁹ and R¹⁰ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R¹¹ and R¹² are each selected from hydrogen, hydroxy, alkoxy, amine, cyano, thiol, halogen, alkyl, substituted alkyl, haloalkyl, heteroalkyl and substituted heteroalkyl;

X is S or O;

n is an integer from 0 to 12; and

or a salt, solvate or hydrate thereof.

In some embodiments, R¹ is hydrogen. In some embodiments, R² is hydrogen. In some embodiments, R⁴ is hydrogen. In some embodiments, R⁵ is hydrogen. In some embodiments, R⁶ is hydrogen. In some embodiments, R⁷ is hydrogen. In some embodiments, R⁸ is hydrogen. In some embodiments, R⁹ is hydrogen. In some embodiments, R¹⁰ is hydrogen. In some embodiments, R¹¹ is hydrogen. In some embodiments, R¹² is hydrogen.

In some embodiments, R⁸ is a C(1-6)alkyl. In some instances, R¹ is a selected from methyl, ethyl, n-propyl, isopropyl, n-butyl, t-butyl, pentyl and hexyl. In certain instances, R¹ is methyl. In some instances, R³ is a substituted alkyl. In some instances, R³ is a haloalkyl. In some instances, R³ is a fluoroalkyl. In some instances, R³ is a trifluoromethyl. In some instances, R³ is halogen. In some instances, R³ is selected from fluorine, chlorine, bromine and iodine. In some instances, R³ is fluorine. In some instances, R³ is bromine. In some instances, R³ is a C(1-6)alkyl. In some instances, R³ is selected from methyl, ethyl, n-propyl, isopropyl, n-butyl, t-butyl, pentyl and hexyl. In certain instances, R³ is ethyl. In some instances, R³ is a substituted C(1-6)alkyl. In some instances, R³ is a thio-substituted C(1-6)alkyl. In some instances, R³ is selected from thio-substituted methyl, thio-substituted ethyl, thio-substituted n-propyl, thio-substituted isopropyl, thio-substituted n-butyl, thio-substituted t-butyl, thio-substituted pentyl and thio-substituted hexyl. In some instances, R³ is methylthio.

In some instances, R² is cyano. In some instances, R² is hydrogen.

In some embodiments, X is O. In other embodiments, X is S.

In certain embodiments, compounds of interest include a compound of formula (II):

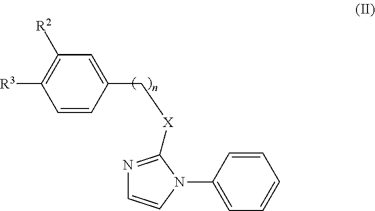

wherein R² and R³ are each independently selected from hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

X is S or O;

n is an integer from 0 to 12; and

or a salt, solvate or hydrate thereof.

In some instances, R³ is a haloalkyl. In some instances, R³ is a fluoroalkyl. In some instances, R³ is a trifluoromethyl. In some instances, R³ is halogen. In some instances, R³ is selected from fluorine, chlorine, bromine and iodine. In some instances, R³ is fluorine. In some instances, R³ is bromine. In some instances, R³ is a C(1-6)alkyl. In some instances, R³ is selected from methyl, ethyl, n-propyl, isopropyl, n-butyl, t-butyl, pentyl and hexyl. In certain instances, R³ is ethyl. In some instances, R³ is a substituted C(1-6)alkyl. In some instances, R³ is a thio-substituted C(1-6)alkyl. In some instances, R³ is selected from thio-substituted methyl, thio-substituted ethyl, thio-substituted n-propyl, thio-substituted isopropyl, thio-substituted n-butyl, thio-substituted t-butyl, thio-substituted pentyl and thio-substituted hexyl. In some instances, R³ is methylthio.

In some instances, R² is cyano.

In some embodiments, X is O. In other embodiments, X is S.

In certain embodiments, compounds of interest include a compound of formula (III):

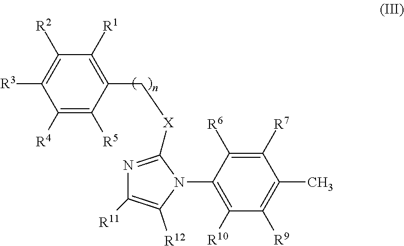

wherein R¹, R², R³, R⁴, R⁵, R⁶, R⁷, R⁹ and R¹⁰ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R¹¹ and R¹² are each selected from hydrogen, hydroxy, alkoxy, amine, cyano, thiol, halogen, alkyl, substituted alkyl, haloalkyl, heteroalkyl and substituted heteroalkyl;

X is S or O;

n is an integer from 0 to 12; and

or a salt, solvate or hydrate thereof.

In some embodiments, R¹ is hydrogen. In some embodiments, R² is hydrogen. In some embodiments, R⁴ is hydrogen. In some embodiments, R⁵ is hydrogen. In some embodiments, R⁶ is hydrogen. In some embodiments, R⁷ is hydrogen. In some embodiments, R⁹ is hydrogen. In some embodiments, R¹⁰ is hydrogen. In some embodiments, R¹¹ is hydrogen. In some embodiments, R¹² is hydrogen.

In some instances, R³ is a substituted alkyl. In some instances, R³ is a haloalkyl. In some instances, R³ is a fluoroalkyl. In some instances, R³ is a trifluoromethyl. In some instances, R³ is halogen. In some instances, R³ is selected from fluorine, chlorine, bromine and iodine. In some instances, R³ is fluorine. In some instances, R³ is bromine. In some instances, R³ is a C(1-6)alkyl. In some instances, R³ is selected from methyl, ethyl, n-propyl, isopropyl, n-butyl, t-butyl, pentyl and hexyl. In certain instances, R³ is ethyl. In some instances, R³ is a substituted C(1-6)alkyl. In some instances, R³ is a thio-substituted C(1-6)alkyl. In some instances, R³ is selected from thio-substituted methyl, thio-substituted ethyl, thio-substituted n-propyl, thio-substituted isopropyl, thio-substituted n-butyl, thio-substituted t-butyl, thio-substituted pentyl and thio-substituted hexyl. In some instances, R³ is methylthio.

In some instances, R² is cyano. In some instances, R² is hydrogen.

In some embodiments, X is O. In other embodiments, X is S.

In certain embodiments, compounds of interest include a compound of formula (IV):

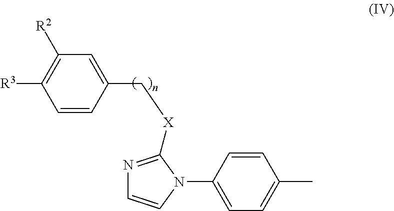

wherein R² and R³ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

X is S or O;

n is an integer from 0 to 12; and

or a salt, solvate or hydrate thereof.

In some instances, R³ is a haloalkyl. In some instances, R³ is a fluoroalkyl. In some instances, R³ is a trifluoromethyl. In some instances, R³ is halogen. In some instances, R³ is selected from fluorine, chlorine, bromine and iodine. In some instances, R³ is fluorine. In some instances, R³ is bromine. In some instances, R³ is a C(1-6)alkyl. In some instances, R³ is selected from methyl, ethyl, n-propyl, isopropyl, n-butyl, t-butyl, pentyl and hexyl. In certain instances, R³ is ethyl. In some instances, R³ is a substituted C(1-6)alkyl. In some instances, R³ is a thio-substituted C(1-6)alkyl. In some instances, R³ is selected from thio-substituted methyl, thio-substituted ethyl, thio-substituted n-propyl, thio-substituted isopropyl, thio-substituted n-butyl, thio-substituted t-butyl, thio-substituted pentyl and thio-substituted hexyl. In some instances, R³ is methylthio.

In some instances, R² is cyano. In some instances, R² is hydrogen.

In some embodiments, X is O. In other embodiments, X is S.

In certain embodiments, the compound is 1-(p-tolyl)-2-((4-(trifluoromethyl)benzyl)thio)-1H-imidazole (Compound 101):

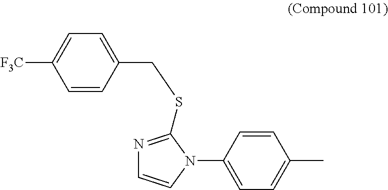 [SMILES: Cc1ccc(-n2ccnc2SCc2ccc(C(F)(F)F)cc2)cc1]

or a pharmaceutically acceptable salt, solvate or hydrate thereof.

In certain embodiments, the compound is 2-((4-fluorobenzyl)thio)-1-phenyl-1H-imidazole (Compound 102):

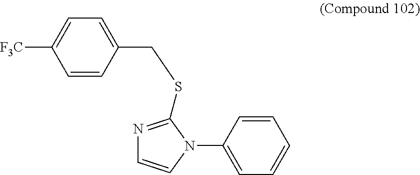 [SMILES: FC(F)(F)c1ccc(CSc2nccn2-c2ccccc2)cc1]

or a pharmaceutically acceptable salt, solvate or hydrate thereof.

In certain embodiments, the compound is 1-(p-tolyl)-2-((4-fluorobenzyl)thio)-1H-imidazole (Compound 102a):

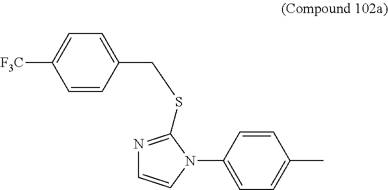 [SMILES: Cc1ccc(-n2ccnc2SCc2ccc(C(F)(F)F)cc2)cc1]

or a pharmaceutically acceptable salt, solvate or hydrate thereof.

In certain embodiments, the compound is 2-((4-(methylthio)benzyl)thio)-1-phenyl-1H-imidazole (Compound 103):

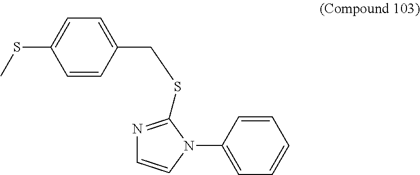 [SMILES: CSc1ccc(CSc2nccn2-c2ccccc2)cc1]

or a pharmaceutically acceptable salt, solvate or hydrate thereof.

In certain embodiments, the compound is 1-(p-tolyl)-2-((4-(methylthio)benzyl)thio)-1H-imidazole (Compound 103a):

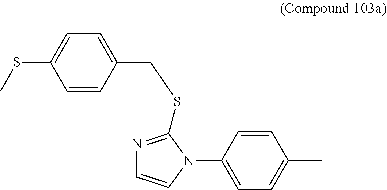

or a pharmaceutically acceptable salt, solvate or hydrate thereof.

In certain embodiments, the compound is 2-fluoro-5-(((1-phenyl-1H-imidazol-2-yl)thio)methyl)benzonitrile (Compound 104):

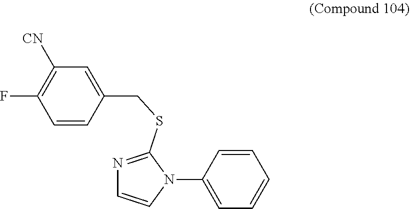

or a pharmaceutically acceptable salt, solvate or hydrate thereof.

In certain embodiments, the compound is 1-(p-tolyl)-2-fluoro-5-(((1H-imidazol-2-yl)thio)methyl)benzonitrile (Compound 104a):

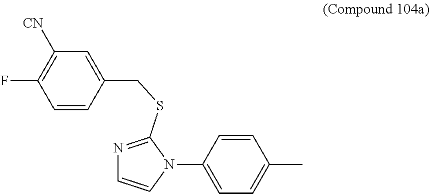

or a pharmaceutically acceptable salt, solvate or hydrate thereof.

Aspects of the disclosure also include methods for modulating or inhibiting human epithelial 15-lipoxygenase-2 (h15-LOX-2) by contacting a cell with an amount of the subject compounds or a pharmaceutically acceptable salt thereof. In some instances, the cell having human epithelial 15-lipoxygenase-2 (h15-LOX-2) is contacted with the compound in vitro. In other instances, the cell having human epithelial 15-lipoxygenase-2 (h15-LOX-2) is contacted with the compound in vivo. In some instances, methods include contacting one or more of the compounds described herein with cells having human epithelial 15-lipoxygenase-2 (h15-LOX-2) in a manner sufficient to modulate the generation of hydroperoxy eicosatetraeneoic acids (HpETEs) in cells (e.g., human cells). In some embodiments, methods include modulating ferroptosis. In some embodiments, methods include modulating ferroptosis in a manner sufficient to reduce the accumulation of hydroperoxy membrane phospholipids in the contacted cells. In some embodiments, methods include modulating eicosanoid mediator biosynthesis from leukotrienes (LTs) to pro-resolving mediator class of lipoxins (LXs). In some embodiments, methods include modulating h15-LOX-2 in a manner sufficient to reduce foam cell formation and atherosclerotic plaque accumulation.

In some embodiments, methods include treating or preventing a human epithelial 15-lipoxygenase-2 mediated disease. In some embodiments, methods include treating or preventing atherosclerotic plaque formation or accumulation. In some embodiments, methods include treating or preventing a cardiovascular disease. In some embodiments, methods include treating or preventing cystic fibrosis lung disease. In some embodiments, methods include treating or preventing a neurodegenerative disease, such as Alzheimer's disease, Parkinson's disease, and Huntington's disease. In some embodiments, methods include administering one or more of the compounds described herein to a subject diagnosed with one or more a cardiovascular disease, cystic fibrosis lung disease and a neurodegenerative disease such as Alzheimer's disease, Parkinson's disease, and Huntington's disease.

### BRIEF DESCRIPTION OF THE FIGURES

FIG. 1 depicts the chemical structures of inhibitor 545091 and inhibitor 536924.

FIG. 2 depicts A representative graph of steady-state inhibition kinetic data for the determination of K_{ic} and Kᵢᵤ of h15-LOX-2 and Compound 105. (A) Dixon plot of the primary data of h15-LOX-2 and Compound 105. The substrate concentrations are 1.5 μM (closed circles), 5 μM (open diamonds), 10 μM (closed triangles), 15 μM (open squares) and 20 μM (closed diamonds). The Dixon replot of slope versus [Inhibitor] yielded a K_{ic} of 0.80 (0.05) M and a Kᵢᵤ of 4.0 (2.6) μM. (y=1844.7x+51.918; R²=0.9896)

FIG. 3 depicts the docking pose of ligands bound to h15-LOX-2 (FIG. 3A) Compound 107, (FIG. 3B) Compound 105, (FIG. 3C) Compound 106, (FIG. 3D) inhibitor 536924, (FIG. 3E) inhibitor 545091.

FIG. 4 depicts electron density in the U-shaped channel. (FIG. 4A) Polder omit map electron density from chain A conuored at 3 a is shown as orange mesh. Mn²⁺ is shown as purple sphere and water molecule as red sphere. (FIG. 4B) Density attributed to a metal-coordinated water molecule was not observed for Chain B. (FIG. 4C and FIG. 4D) Omit map |Fo-Fc| electron density contoured at 3 a is shown as green mesh for chain A and B, respectively.

FIG. 5 depicts inhibitor 536924 in the U-shaped channel. Surface rendering in the cavities and pockets only mode from Pymol of chain A of the 15-LOX-2 LM structure (71af.pdb) is shown as grey. The Mn²⁺ is shown as purple sphere and water molecule as red sphere. Inhibitor 536924 (C, pink) binds in the U-shaped channel and interacts with the water that coordinates the 6^{th} position of the metal.

FIG. 6 depicts metal coordination sphere. Electron density |2Fo-Fc| is shown as blue mesh and contoured at 1 σ from the software Coot {ref PMID 15572765}. In view are the C-terminal carboxylate from Ile 676 along with His 553 and His 373 coordinating the Mn²⁺ Inhibitor 536924 binds above the metal-coordination sphere. Positive Omit map density |Fo-Fc| contoured at 3 σ is shown as green mesh where a water molecule should fill the 6^{th}, position of the metal.

FIG. 7 depicts the HDX-MS properties of 15-LOX-2 isozyme-specific inhibitors. (FIG. 7A) HDX-MS behavior at 2 h and 25° C. of h15-LOX-2 peptides in the absence of an inhibitor is mapped onto the crystal structure (PDB entry, 4NRE). The coloring is defined by the spectrum bar. Black coloring represents uncovered regions. The PLAT domain is highlighted by cyan outline and the helix α2 is highlighted by the green box. (FIG. 7B, FIG. 7C) presents the impact of isozyme selective inhibitors 536924 and Compound 101, on the HDX-MS properties of 15-LOX-2. The effect is localized to the helix α2 of 15-LOX-2 and the peptides are colored as follows: chartreuse, 173-184; marine, 184-191; and salmon, 192-206.

FIG. 8 depicts HDX-MS traces (25° C.) of helix α2 peptide, comparing data reported from Droege 2019 (red) and data collected herein (black) in the absence of inhibitor (A). In (B), HDX-MS data for helix α2 are compared that were collected on human 15-LOX-2 purified from E. coli in the Offenbacher lab (black) and from SF9 insect cells in the Holman lab (green).

FIG. 9 depicts evolution of time-dependent exchange of WT 15-LOX-2 and SLO-1 at 25° C.

FIG. 10 depicts alternative peptides for HDX properties of 15-LOX-2 helix α2. The colors represent: black, 15LOX2 alone; gray, 15LOX2/536924; green, 15LOX2/Compound 105.

FIG. 11 depicts EC₅₀ values for the newly discovered inhibitors targeting h15-LOX-2 expressed in HEK293T cells. All experiments were conducted in triplicate and with 10 μM exogenous AA added

FIG. 12 depicts binding of inhibitor 536924, inhibitor 545091, Compound 105, Compound 106 and Compound 107 binding in the U-shaped channel of h15-LOX-2 in similar binding poses.

### DEFINITIONS

The following terms have the following meaning unless otherwise indicated. Any undefined terms have their art recognized meanings.

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³¹ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

The term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the numbers of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated 7L electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, 3-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), —M, —R⁶⁰, —O—, ═O, —OR⁶⁰, —SR⁶⁰, —S—, ═S, —NR^{6O}R⁶¹═NR⁶⁰—CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹,—C(O)O—, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include —M, —R⁶⁰, ═O, —OR⁶⁰, —SR⁶⁰, —S—, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O—, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹,—C(O)O—, —NR⁶²C(O)NR⁶⁰R⁶¹. In certain embodiments, substituents include —M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰, R⁶¹,—C(O)O. In certain embodiments, substituents include —M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰,—C(O)O, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

“Pharmaceutically acceptable carrier” refers to a diluent, adjuvant, excipient or vehicle with, or in which a compound is administered.

### DETAILED DESCRIPTION

Compounds for inhibiting human epithelial 15-lipoxygenase-2 (h15-LOX-2) are provided. Compounds according to certain embodiments modulate ferroptosis and generation of hydroperoxy eicosatetraeneoic acids (HpETEs). In some embodiments, compounds described herein modulate eicosanoid mediator biosynthesis from leukotrienes (LTs) to pro-resolving mediator class of lipoxins (LXs). Methods for treating or preventing a human epithelial 15-lipoxygenase-2 (h15-LOX-2)-mediated disease are also provided. Compositions for practicing the subject methods are also described.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the compounds and methods have or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

Reference will now be made in detail to various embodiments. It will be understood that the invention is not limited to these embodiments. To the contrary, it is intended to cover alternatives, modifications, and equivalents as may be included within the spirit and scope of the allowed claims.

Compounds for Modulating Human Epithelial 15-lipoxygenase-2

In some embodiments, compounds of the present disclosure include a compound of formula (I):

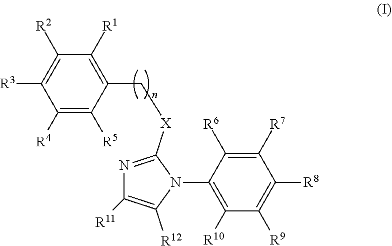

wherein R¹, R², R³, R⁴, R⁵, R⁶, R⁷, R⁸, R⁹ and R¹⁰ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R¹¹ and R¹² are each selected from hydrogen, hydroxy, alkoxy, amine, cyano, thiol, halogen, alkyl, substituted alkyl, haloalkyl, heteroalkyl and substituted heteroalkyl;

X is S or O;

n is an integer from 0 to 12; and

or a salt, solvate or hydrate thereof.

In embodiments, “salts” of the compounds of the present disclosure may include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

The term “solvate” as used herein refers to a complex or aggregate formed by one or more molecules of a solute, e.g. a compound of Formula (I) or a salt thereof, and one or more molecules of a solvent. Such solvates may be crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some instances, R¹ is hydrogen. In some instances, R¹ is a C(1-6)alkyl. In some instances, R¹ is selected from methyl, ethyl, n-propyl, isopropyl, n-butyl, t-butyl, pentyl and hexyl. In some instances, R¹ is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R¹ is a hetero C(1-6)alkyl. In some instances, R¹ is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R¹ is selected from hydroxy, alkoxy, amine, cyano, thiol and halogen. In certain instances, the halogen is selected from fluorine, chlorine, bromine and iodine.

In some instances, R² is hydrogen. In some instances, R² is cyano. In some instances, R² is a C(1-6)alkyl. In some instances, R² is selected from methyl, ethyl, n-propyl, isopropyl, n-butyl, t-butyl, pentyl and hexyl. In some instances, R² is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R² is a hetero C(1-6)alkyl. In some instances, R² is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R² is selected from hydroxy, alkoxy, amine, thiol and halogen. In certain instances, the halogen is selected from fluorine, chlorine, bromine and iodine.

In some instances, R³ is a substituted alkyl. In some instances, R³ is a haloalkyl. In some instances, R³ is a fluoroalkyl. In some instances, R³ is a trifluoromethyl. In some instances, R³ is halogen. In some instances, R³ is selected from fluorine, chlorine, bromine and iodine. In some instances, R³ is fluorine. In some instances, R³ is bromine. In some instances, R³ is a C(1-6)alkyl. In some instances, R³ is selected from methyl, ethyl, n-propyl, isopropyl, n-butyl, t-butyl, pentyl and hexyl. In certain instances, R³ is ethyl. In some instances, R³ is a substituted C(1-6)alkyl. In some instances, R³ is a thio-substituted C(1-6)alkyl. In some instances, R³ is selected from thio-substituted methyl, thio-substituted ethyl, thio-substituted n-propyl, thio-substituted isopropyl, thio-substituted n-butyl, thio-substituted t-butyl, thio-substituted pentyl and thio-substituted hexyl. In some instances, R³ is methylthio.

In some instances, R⁴ is hydrogen. In some instances, R⁴ is a C(1-6)alkyl. In some instances, R⁴ is selected from methyl, ethyl, n-propyl, isopropyl, n-butyl, t-butyl, pentyl and hexyl. In some instances, R⁴ is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R⁴ is a hetero C(1-6)alkyl. In some instances, R⁴ is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R⁴ is selected from hydroxy, alkoxy, amine, cyano, thiol and halogen. In certain instances, the halogen is selected from fluorine, chlorine, bromine and iodine.

In some instances, R^{s} is hydrogen. In some instances, R^{s} is a C(1-6)alkyl. In some instances, R^{s} is selected from methyl, ethyl, n-propyl, isopropyl, n-butyl, t-butyl, pentyl and hexyl. In some instances, R^{s} is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R^{s} is a hetero C(1-6)alkyl. In some instances, R^{s} is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R^{s} is selected from hydroxy, alkoxy, amine, cyano, thiol and halogen. In certain instances, the halogen is selected from fluorine, chlorine, bromine and iodine.

In some instances, R⁶ is hydrogen. In some instances, R⁶ is a C(1-6)alkyl. In some instances, R⁶ is selected from methyl, ethyl, n-propyl, isopropyl, n-butyl, t-butyl, pentyl and hexyl. In some instances, R⁶ is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R⁶ is a hetero C(1-6)alkyl. In some instances, R⁶ is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R⁶ is selected from hydroxy, alkoxy, amine, cyano, thiol and halogen. In certain instances, the halogen is selected from fluorine, chlorine, bromine and iodine.

In some instances, R⁷ is hydrogen. In some instances, R⁷ is a C(1-6)alkyl. In some instances, R⁷ is selected from methyl, ethyl, n-propyl, isopropyl, n-butyl, t-butyl, pentyl and hexyl. In some instances, R⁷ is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R⁷ is a hetero C(1-6)alkyl. In some instances, R⁷ is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R⁷ is selected from hydroxy, alkoxy, amine, cyano, thiol and halogen. In certain instances, the halogen is selected from fluorine, chlorine, bromine and iodine.

In some embodiments, R¹ is a C(1-6)alkyl. In some instances, R¹ is a selected from methyl, ethyl, n-propyl, isopropyl, n-butyl, t-butyl, pentyl and hexyl. In certain instances, R¹ is methyl. In some instances, R¹ is hydrogen. In some instances, R¹ is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R¹ is a hetero C(1-6)alkyl. In some instances, R¹ is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R¹ is selected from hydroxy, alkoxy, amine, cyano, thiol and halogen. In certain instances, the halogen is selected from fluorine, chlorine, bromine and iodine.

In some instances, R⁹ is hydrogen. In some instances, R⁹ is a C(1-6)alkyl. In some instances, R⁹ is selected from methyl, ethyl, n-propyl, isopropyl, n-butyl, t-butyl, pentyl and hexyl. In some instances, R⁹ is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R⁹ is a hetero C(1-6)alkyl. In some instances, R⁹ is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R⁹ is selected from hydroxy, alkoxy, amine, cyano, thiol and halogen. In certain instances, the halogen is selected from fluorine, chlorine, bromine and iodine.

In some instances, R¹⁰ is hydrogen. In some instances, R¹⁰ is a C(1-6)alkyl. In some instances, R¹⁰ is selected from methyl, ethyl, n-propyl, isopropyl, n-butyl, t-butyl, pentyl and hexyl. In some instances, R¹⁰ is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R¹⁰ is a hetero C(1-6)alkyl.

In some instances, R¹⁰ is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R¹⁰ is selected from hydroxy, alkoxy, amine, cyano, thiol and halogen. In certain instances, the halogen is selected from fluorine, chlorine, bromine and iodine.

In some instances, R¹¹ is hydrogen. In some instances, R¹¹ is a C(1-6)alkyl. In some instances, R¹¹ is selected from methyl, ethyl, n-propyl, isopropyl, n-butyl, t-butyl, pentyl and hexyl. In some instances, R¹¹ is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R¹¹ is a hetero C(1-6)alkyl. In some instances, R¹¹ is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R¹¹ is selected from hydroxy, alkoxy, amine, cyano, thiol and halogen. In certain instances, the halogen is selected from fluorine, chlorine, bromine and iodine.

In some instances, R¹² is hydrogen. In some instances, R¹² is a C(1-6)alkyl. In some instances, R¹² is selected from methyl, ethyl, n-propyl, isopropyl, n-butyl, t-butyl, pentyl and hexyl. In some instances, R¹² is a substituted C(1-6)alkyl, such as as substituted methyl, substituted ethyl, substituted n-propyl, substituted isopropyl, substituted n-butyl, substituted t-butyl, substituted pentyl and substituted hexyl. In some instances, R¹¹ is a hetero C(1-6)alkyl.

In some instances, R¹¹ is selected from a cycloalkyl, a substituted cycloalkyl, a heterocycloalkyl, a substituted heterocycloalkyl, an aryl, a substituted aryl, an arylalkyl, a substituted arylalkyl, a heteroaryl, a substituted heteroaryl, a heteroarylalkyl, and a substituted heteroarylalkyl. In some instances, R¹¹ is selected from hydroxy, alkoxy, amine, cyano, thiol and halogen. In certain instances, the halogen is selected from fluorine, chlorine, bromine and iodine.

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12.

In certain embodiments, X is O. In certain embodiments, X is S.

In certain embodiments, compounds of interest include a compound of formula (II):

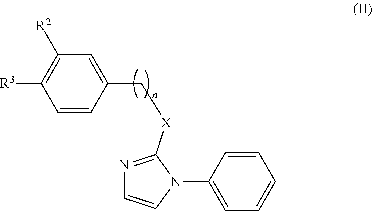

wherein R² and R³ are each independently selected from hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

X is S or O;

n is an integer from 0 to 12; and

or a salt, solvate or hydrate thereof.

In some instances, R³ is a haloalkyl. In some instances, R³ is a fluoroalkyl. In some instances, R³ is a trifluoromethyl. In some instances, R³ is halogen. In some instances, R³ is selected from fluorine, chlorine, bromine and iodine. In some instances, R³ is fluorine. In some instances, R³ is bromine. In some instances, R³ is a C(1-6)alkyl. In some instances, R³ is selected from methyl, ethyl, n-propyl, isopropyl, n-butyl, t-butyl, pentyl and hexyl. In certain instances, R³ is ethyl. In some instances, R³ is a substituted C(1-6)alkyl. In some instances, R³ is a thio-substituted C(1-6)alkyl. In some instances, R³ is selected from thio-substituted methyl, thio-substituted ethyl, thio-substituted n-propyl, thio-substituted isopropyl, thio-substituted n-butyl, thio-substituted t-butyl, thio-substituted pentyl and thio-substituted hexyl. In some instances, R³ is methylthio.

In some instances, R² is cyano.

In some embodiments, X is O. In other embodiments, X is S.

In certain embodiments, compounds of interest include a compound of formula (III):

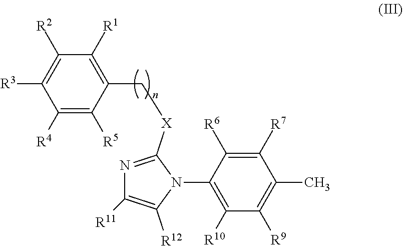

wherein R¹, R², R³, R⁴, R⁵, R⁶, R⁷, R⁹ and R¹⁰ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R¹¹ and R¹² are each selected from hydrogen, hydroxy, alkoxy, amine, cyano, thiol, halogen, alkyl, substituted alkyl, haloalkyl, heteroalkyl and substituted heteroalkyl;

X is S or O;

n is an integer from 0 to 12; and

or a salt, solvate or hydrate thereof.

In some embodiments, R¹ is hydrogen. In some embodiments, R² is hydrogen. In some embodiments, R⁴ is hydrogen. In some embodiments, R⁵ is hydrogen. In some embodiments, R⁶ is hydrogen. In some embodiments, R⁷ is hydrogen. In some embodiments, R⁹ is hydrogen. In some embodiments, R¹⁰ is hydrogen. In some embodiments, R¹¹ is hydrogen. In some embodiments, R¹² is hydrogen.

In some instances, R³ is a substituted alkyl. In some instances, R³ is a haloalkyl. In some instances, R³ is a fluoroalkyl. In some instances, R³ is a trifluoromethyl. In some instances, R³ is halogen. In some instances, R³ is selected from fluorine, chlorine, bromine and iodine. In some instances, R³ is fluorine. In some instances, R³ is bromine. In some instances, R³ is a C(1-6)alkyl. In some instances, R³ is selected from methyl, ethyl, n-propyl, isopropyl, n-butyl, t-butyl, pentyl and hexyl. In certain instances, R³ is ethyl. In some instances, R³ is a substituted C(1-6)alkyl. In some instances, R³ is a thio-substituted C(1-6)alkyl. In some instances, R³ is selected from thio-substituted methyl, thio-substituted ethyl, thio-substituted n-propyl, thio-substituted isopropyl, thio-substituted n-butyl, thio-substituted t-butyl, thio-substituted pentyl and thio-substituted hexyl. In some instances, R³ is methylthio.

In some instances, R² is cyano. In some instances, R² is hydrogen.

In some embodiments, X is O. In other embodiments, X is S.

In certain embodiments, compounds of interest include a compound of formula (IV):

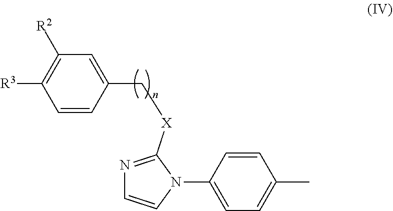

wherein R² and R³ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

X is S or O;

n is an integer from 0 to 12; and

or a salt, solvate or hydrate thereof.

In some instances, R³ is a haloalkyl. In some instances, R³ is a fluoroalkyl. In some instances, R³ is a trifluoromethyl. In some instances, R³ is halogen. In some instances, R³ is selected from fluorine, chlorine, bromine and iodine. In some instances, R³ is fluorine. In some instances, R³ is bromine. In some instances, R³ is a C(1-6)alkyl. In some instances, R³ is selected from methyl, ethyl, n-propyl, isopropyl, n-butyl, t-butyl, pentyl and hexyl. In certain instances, R³ is ethyl. In some instances, R³ is a substituted C(1-6)alkyl. In some instances, R³ is a thio-substituted C(1-6)alkyl. In some instances, R³ is selected from thio-substituted methyl, thio-substituted ethyl, thio-substituted n-propyl, thio-substituted isopropyl, thio-substituted n-butyl, thio-substituted t-butyl, thio-substituted pentyl and thio-substituted hexyl. In some instances, R³ is methylthio.

In some instances, R² is cyano. In some instances, R² is hydrogen.

In some embodiments, X is O. In other embodiments, X is S.

In certain embodiments, the compound is 1-(p-tolyl)-2-((4-(trifluoromethyl)benzyl)thio)-1H-imidazole (Compound 101):

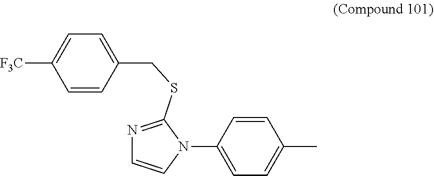 [SMILES: Cc1ccc(-n2ccnc2SCc2ccc(C(F)(F)F)cc2)cc1]

or a pharmaceutically acceptable salt, solvate or hydrate thereof.

In certain embodiments, the compound is 2-((4-fluorobenzyl)thio)-1-phenyl-1H-imidazole (Compound 102):

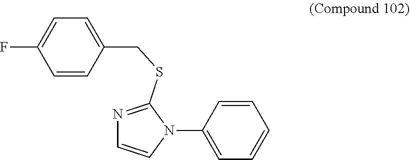 [SMILES: Fc1ccc(CSc2nccn2-c2ccccc2)cc1]

or a pharmaceutically acceptable salt, solvate or hydrate thereof.

In certain embodiments, the compound is 1-(p-tolyl)-2-((4-fluorobenzyl)thio)-1H-imidazole (Compound 102a):

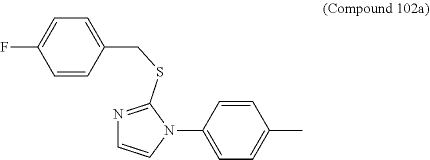 [SMILES: Cc1ccc(-n2ccnc2SCc2ccc(F)cc2)cc1]

or a pharmaceutically acceptable salt, solvate or hydrate thereof.

In certain embodiments, the compound is 2-((4-(methylthio)benzyl)thio)-1-phenyl-1H-imidazole (Compound 103):

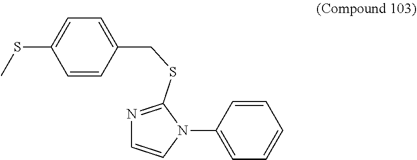 [SMILES: CSc1ccc(CSc2nccn2-c2ccccc2)cc1]

or a pharmaceutically acceptable salt, solvate or hydrate thereof.

In certain embodiments, the compound is 1-(p-tolyl)-2-((4-(methylthio)benzyl)thio)-1H-imidazole (Compound 103a):

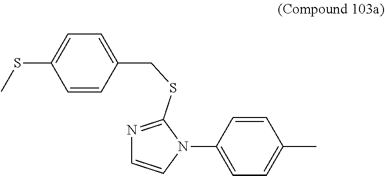

or a pharmaceutically acceptable salt, solvate or hydrate thereof.

In certain embodiments, the compound is 2-fluoro-5-(((1-phenyl-1H-imidazol-2-yl)thio)methyl)benzonitrile (Compound 104):

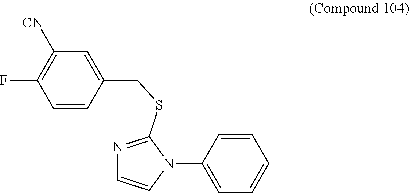

or a pharmaceutically acceptable salt, solvate or hydrate thereof.

In certain embodiments, the compound is 1-(p-tolyl)-2-fluoro-5-(((1H-imidazol-2-yl)thio)methyl)benzonitrile (Compound 104a):

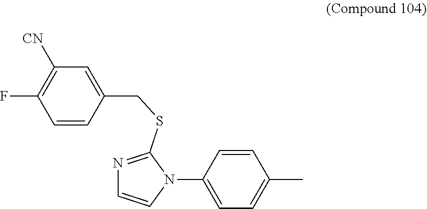

or a pharmaceutically acceptable salt, solvate or hydrate thereof.

Aspects of the present disclosure also include compositions having a pharmaceutically acceptable carrier and one or more of the compounds described above. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

The compounds may be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In certain embodiments, the conjugate compounds are formulated for injection. For example, compositions of interest may be formulated for intravenous or intraperitoneal administration.

In pharmaceutical dosage forms, the compounds may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interst further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Where the composition is formulated for injection, the compounds may be formulated by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Although the dosage used in treating a subject will vary depending on the clinical goals to be achieved, a suitable dosage range of the compound is one which provides up to about 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the severity of the symptoms and the susceptibility of the subject to side effects.

In some embodiments, a single dose of the compound is administered. In other embodiments, multiple doses of the compound are administered. Where multiple doses are administered over a period of time, the compound may be administered, e.g., twice daily (qid), daily (qd), every other day (qod), every third day, three times per week (tiw), or twice per week (biw) over a period of time. For example, the compound may be administered qid, qd, qod, tiw, or biw over a period of from one day to about 2 years or more. For example, the compound may be administered at any of the aforementioned frequencies for one week, two weeks, one month, two months, six months, one year, or two years, or more, depending on various factors.

Dose units of the present disclosure can be made using manufacturing methods available in the art and can be of a variety of forms suitable for injection (including topical, intracisternal, intrathecal, intravenous, intramuscular, subcutaneous and dermal) administration, for example as a solution, suspension, solution, lyophilate or emulsion. The dose unit can contain components conventional in pharmaceutical preparations, e.g. one or more carriers, binders, lubricants, excipients (e.g., to impart controlled release characteristics), pH modifiers, coloring agents or further active agents.

Dose units can comprise components in any relative amounts. For example, dose units can be from about 0.1% to 99% by weight of active ingredients (i.e., compounds described herein) per total weight of dose unit. In some embodiments, dose units can be from 10% to 50%, from 20% to 40%, or about 30% by weight of active ingredients per total weight dose unit.

Methods for Modulating Human Epithelial 15-lipoxygenase-2 (h15-LOX-2)

As summarized above, aspects of the present disclosure also modulating or inhibiting epithelial 15-lipoxygenase-2. In some embodiments, methods include contacting a cell having human epithelial 15-lipoxygenase-2 (hl5-LOX-2) with one or more of the compounds described herein in vitro. In other embodiments, methods include contacting a cell having human epithelial 15-lipoxygenase-2 (hl5-LOX-2) with one or more of the compounds described herein in vivo (e.g., by administering to a subject as described in greater detail below). In still other embodiments a cell having human epithelial 15-lipoxygenase-2 (hl5-LOX-2) is contacted ex vivo. In some embodiments, methods include decreasing or reducing hl5-LOX-2 acitivity, such as reducing h15-LOX-2 acitivity by 1% or more, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more.

In some embodiments, the subject methods include modulating the generation of hydroperoxy eicosatetraeneoic acids (HpETEs) in cells, such as where generation of hydroperoxy eicosatetraeneoic acids (e.g., HpETE-PE) is reduced by 1% or more, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more. In certain embodiments, methods include modulating ferroptosis. In certain instances, methods include contacting one or more of the compounds described herein with cells having human epithelial 15-lipoxygenase-2 (hl5-LOX-2) in a manner sufficient to reduce the accumulation of hydroperoxy membrane phospholipids in the contacted cells by 1% or more, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more.

In other embodiments, methods include modulating or reducing modulating eicosanoid mediator biosynthesis from leukotrienes (LTs) to pro-resolving mediator class of lipoxins (LXs).

In some embodiments, methods include modulating hl5-LOX-2 in a manner sufficient to reduce foam cell formation and atherosclerotic plaque accumulation, such as by 1% or more, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more.

In some instances, methods include treating or preventing a human epithelial 15-lipoxygenase-2 mediated disease. The term “treat” or “treatment” of any condition, refers, in certain embodiments, to ameliorating the condition (i.e., arresting or reducing the development of the condition). In certain embodiments “treating” or “treatment” refers to ameliorating at least one physical parameter, which may not be discernible by the patient. In certain embodiments, “treating” or “treatment” refers to inhibiting the condition, either physically, (e.g., stabilization of a discernible symptom), physiologically, (e.g., stabilization of a physical parameter), or both. In certain embodiments, “treating” or “treatment” refers to delaying the onset of the condition. The term “therapeutically effective amount” is used herein to refer to the amount of a compound that, when administered to a patient for preventing or treating a condition is sufficient to effect such treatment. The “therapeutically effective amount” will vary depending on the compound, the condition and its severity and the age, weight, etc., of the patient.

In practicing the subject methods, a therapeutically effective amount of one or more of the compounds disclosed herein is administered to a subject sufficient to treat or prevent the human epithelial 15-lipoxygenase-2 mediated diseases. In embodiments, the term “subject” is meant the person or organism to which the compound is administered. As such, subjects of the present disclosure may include but are not limited to mammals, e.g., humans and other primates, such as chimpanzees and other apes and monkey species, dogs, rabbits, cats and other domesticated pets; and the like, where in certain embodiments the subject are humans. The term “subject” is also meant to include a person or organism of any age, weight or other physical characteristic, where the subjects may be an adult, a child, an infant or a newborn.

In some instances, the human epithelial 15-lipoxygenase-2 mediated disease is a cardiovascular disease or condition such as atherosclerotic plaque formation or accumulation. In some embodiments, methods include treating a subject for cystic fibrosis lung disease. In some embodiments, methods include treating a subject for neurodegenerative disease. In certain instances, methods include treating a subject for Alzheimer's disease. In certain instances, methods include treating a subject for Parkinson's disease. In certain instances, methods include treating a subject for Huntington's disease. In certain embodiments, methods further include diagnosing the subject as having one or more a cardiovascular disease, cystic fibrosis lung disease or a neurodegenerative disease such as Alzheimer's disease, Parkinson's disease, and Huntington's disease.

Compounds as described herein may be administered to a subject by any convenient protocol, including, but not limited, to intraperitoneally, topically, orally, sublingually, parenterally, intravenously, vaginally, rectally as well as by transdermal protocols. In certain embodiments, the subject compounds are administered by intravenous injection. In certain embodiments, the subject compounds are administered by intraperitoneal injection.

Depending on the condition being treated, the amount of compound administered to the subject may vary, such as ranging from about 0.0001 mg/day to about 10,000 mg/day, such as from about 0.001 mg/day to about 9000 mg/day, such as from 0.01 mg/day to about 8000 mg/day, such as from about 0.1 mg/day to about 7000 mg/day, such as from about 1 mg/day to about 6000 mg/day, including from about 5 mg/day to about 5000 mg/day. Each dosage of the compound or pharmaceutically acceptable salt administered to the subject may vary ranging from about 1 mg/kg to about 1000 mg/kg, such as from about 2 mg/kg to about 900 mg/kg, such as from about 3 mg/kg to about 800 mg/kg, such as from about 4 mg/kg to about 700 mg/kg, such as from 5 mg/kg to about 600 mg/kg, such as from 6 mg/kg to about 500 mg/kg, such as from 7 mg/kg to about 400 mg/kg, such as from about 8 mg/kg to about 300 mg/kg, such as from about 9 mg/kg to about 200 mg/kg and including from about 10 mg/kg to about 100 mg/kg.

In certain embodiments, protocols may include multiple dosage intervals. By “multiple dosage intervals” is meant that two or more dosages of the compound is administered to the subject in a sequential manner. In practicing methods of the present disclosure, treatment regimens may include two or more dosage intervals, such as three or more dosage intervals, such as four or more dosage intervals, such as five or more dosage intervals, including ten or more dosage intervals. The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer.

The cycles of drug administration may be repeated for 1, 2, 3, 4, 5, 6, 7, 8 or more than 8 dosage cycles, for a total period of 6 months or 1 year or 2 years or 3 years or 4 years or more. In certain embodiments, one or more of the subject compounds are administered for the rest of the subject's lifetime.

In certain embodiments, compounds of the present disclosure can be administered prior to, concurrent with, or subsequent to other therapeutic agents for treating the same or an unrelated condition. If provided at the same time as another therapeutic agent, the present compounds may be administered in the same or in a different composition. Thus, the compounds of interest and other therapeutic agents can be administered to the subject by way of concurrent therapy. By “concurrent therapy” is intended administration to a subject such that the therapeutic effect of the combination of the substances is caused in the subject undergoing therapy. For example, concurrent therapy may be achieved by administering the compounds of the present disclosure with a pharmaceutical composition having at least one other agent, such as an anti-inflammatory agent, immunosuppressant, steroid, analgesic, anesthetic, antihypertensive, chemotherapeutic, among other types of therapeutics, which in combination make up a therapeutically effective dose, according to a particular dosing regimen. Administration of the separate pharmaceutical compositions can be performed simultaneously or at different times (i.e., sequentially, in either order, on the same day, or on different days), so long as the therapeutic effect of the combination of these substances is caused in the subject undergoing therapy.

Where the compounds of the present disclosure is administered concurrently with a second therapeutic agent to treat the same condition (e.g., an anti-atherosclerotic drug, etc.) the weight ratio of the subject compound to second therapeutic agent may range from 1:2 and 1:2.5; 1:2.5 and 1:3; 1:3 and 1:3.5 1:3.5 and 1:4; 1:4 and 1:4.5; 1:4.5 and 1:5; 1:5 and 1:10; and 1:10 and 1:25 or a range thereof. For example, the weight ratio of the subject compound to second therapeutic agent may range between 1:1 and 1:5; 1:5 and 1:10; 1:10 and 1:15; or 1:15 and 1:25. Alternatively, the weight ratio of the second therapeutic agent to the subject compound ranges between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; and 10:1 and 25:1 or a range thereof. For example, the ratio of the second therapeutic agent the subject compound may range between 1:1 and 5:1; 5:1 and 10:1; 10:1 and 15:1; or 15:1 and 25:1.

Aspects, including embodiments, of the subject matter described herein may be beneficial alone or in combination, with one or more other aspects or embodiments. Without limiting the description, certain non-limiting aspects of the disclosure numbered 1-37 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A compound of formula I:

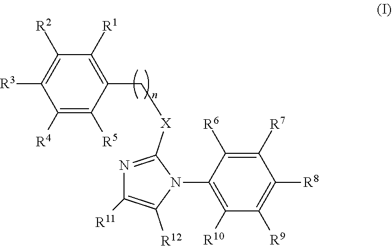

wherein R¹, R², R³, R⁴, R⁵, R⁶, R⁷, R⁸, R⁹ and R¹⁰ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹¹ and R¹² are each selected from hydrogen, hydroxy, alkoxy, amine, cyano, thiol, halogen, alkyl, substituted alkyl, haloalkyl, heteroalkyl and substituted heteroalkyl;
X is S or O; and
n is an integer from 0 to 12,
or a salt, solvate or hydrate thereof.
2. The compound according to 1, wherein X is S.
3. The compound according to any one of 1-2, wherein n is an integer from 1-4.
4. The compound according to 3, wherein n is 1.
5. The compound according to any one of 1-4, wherein R¹¹ and R¹² are each hydrogen.
6. The compound according to any one of 1-5, wherein R¹ is a C(1-6)alkyl.
7. The compound according to 6, wherein R¹ is selected from methyl, ethyl, n-propyl, isopropyl, n-butyl, t-butyl, pentyl and hexyl.
8. The compound according to any one of 6-7, wherein R¹ is methyl.
9. The compound according to any one of 1-8, wherein R³ is a haloalkyl.
10. The compound according to 9, wherein R³ is trifluoromethyl.
11. The compound according to any one of 1-8, wherein R³ is halogen.
12. The compound according to 11, wherein R³ is selected from fluorine, chlorine, bromine and iodine.
13. The compound according to 12, wherein R³ is fluorine or bromine.
14. The compound according to any one of 1-8, wherein R³ is C(1-6)alkyl.
15. The compound according to 14, wherein R³ is selected from methyl, ethyl, n-propyl, isopropyl, n-butyl, t-butyl, pentyl and hexyl.
16. The compound according to any one of 14-15, wherein R³ is ethyl.
17. The compound according to any one of 1-8, wherein R³ is substituted C(1-6)alkyl.
18. The compound according to 17, wherein R³ is thio-substituted C(1-6)alkyl.
19. The compound according to 18, wherein R³ is methylthio.
20. The compound according to any one of 1-19, wherein R² is cyano.
21. The compound according to any one of 1-20, wherein the compound is 1-(p-tolyl)-2-((4-(trifluoromethyl)benzyl)thio)-1H-imidazole (Compound 101):

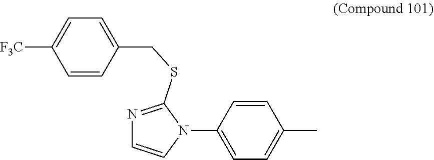 [SMILES: Cc1ccc(-n2ccnc2SCc2ccc(C(F)(F)F)cc2)cc1]

or a salt, solvate or hydrate thereof.
22. The compound according to any one of 1-20, wherein the compound is 2-((4-fluorobenzyl)thio)-1-phenyl-1H-imidazole (Compound 102):

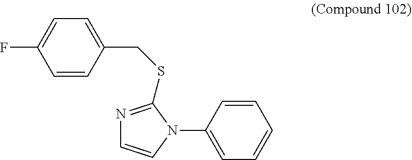 [SMILES: Fc1ccc(CSc2nccn2-c2ccccc2)cc1]

or a salt, solvate or hydrate thereof.
23. The compound according to any one of 1-20, wherein the compound 1-(p-tolyl)-2-((4-fluorobenzyl)thio)-1H-imidazole (Compound 102a):

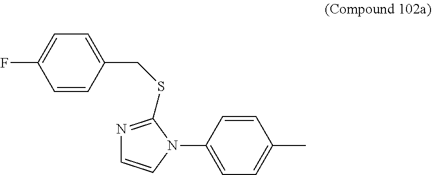 [SMILES: Cc1ccc(-n2ccnc2SCc2ccc(F)cc2)cc1]

or a salt, solvate or hydrate thereof.
24. The compound according to any one of 1-20, wherein the compound is 2-((4-(methylthio)benzyl)thio)-1-phenyl-1H-imidazole (Compound 103):

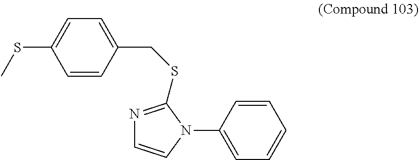 [SMILES: CSc1ccc(CSc2nccn2-c2ccccc2)cc1]

or a salt, solvate or hydrate thereof
25. The compound according to any one of claims 1-20, wherein the compound is 1-(p-tolyl)-2-((4-(methylthio)benzyl)thio)-1H-imidazole (Compound 103a):

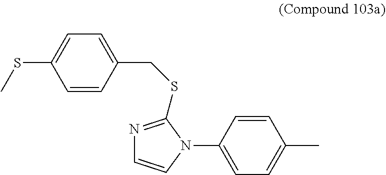

or a salt, solvate or hydrate thereof.
26. The compound according to any one of 1-20, wherein the compound is 2-fluoro-5-(((1-phenyl-1H-imidazol-2-yl)thio)methyl)benzonitrile (Compound 104):

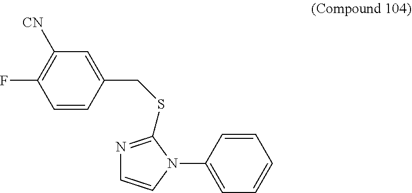

or a salt, solvate or hydrate thereof.
27. The compound according to any one of 1-20, wherein the compound is 1-(p-tolyl)-2-fluoro-5-(((1H-imidazol-2-yl)thio)methyl)benzonitrile (Compound 104a):

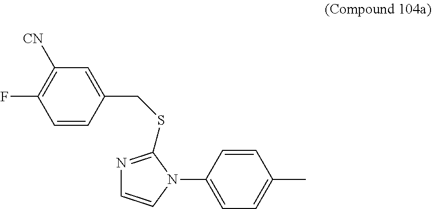

or a salt, solvate or hydrate thereof.
28. A composition comprising:
a compound according to any one of 1-27; and
a pharmaceutically acceptable excipient.
29. A method for inhibiting human epithelial 15-(S)-lipoxygenase, the method comprising contacting a cell with a compound according to any one of 1-27 or a composition according to 28.
30. A method comprising administering to a subject in need thereof a therapeutically effective amount of a compound according to any one of 1-27 or a composition according to claim 28.
31. The method according to 30, wherein the subject is diagnosed with a cardiovascular disease.
32. The method according to 31, wherein the subject is diagnosed with atherosclerosis.
33. The method according to 30, wherein the subject is diagnosed with cystic fibrosis.
34. The method according to 30, wherein the subject is diagnosed with a neurodegenerative disease.
35. The method according to 34, wherein the subject is diagnosed with Alzheimer's disease.
36. The method according to 34, wherein the subject is diagnosed with Parkinson's disease.
37. The method according to 34, wherein the subject is diagnosed with Huntington's disease.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the compounds of the present disclosure, it may be necessary and/or desirable to protect sensitive or reactive groups on any of the molecules concerned. This can be achieved by means of conventional protecting groups as described in standard works, such as T. W. Greene and P. G. M. Wuts, “Protective Groups in Organic Synthesis”, Fourth edition, Wiley, New York 2006. The protecting groups can be removed at a convenient subsequent stage using methods known from the art.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

The nomenclature used herein to name the subject compounds is illustrated in the Examples herein. When possible, this nomenclature has generally been derived using the commercially-available AutoNom software (MDL, San Leandro, Calif).

### Materials and Methods

### Chemicals

Fatty acids used in this study were purchased from Nu Chek Prep, Inc. (MN, USA). All other solvents and chemicals were reagent grade or better and were used as purchased without further purification.

### Synthesis of Inhibitors of human epithelial 15-lipoxygenase-2 (h15-LOX-2)

All air or moisture sensitive reactions were performed under positive pressure of nitrogen with oven-dried glassware. Chemical reagents and anhydrous solvents were obtained from commercial sources and used as—is. The analog for assay has purity greater than 95% and ¹H and ¹³C NMR spectra were recorded on Bruker Avance III HD 500 MHz NMR spectrometer.

General Synthetic Procedures. The synthesis of the compounds described herein was achieved with the following steps (Scheme 1).

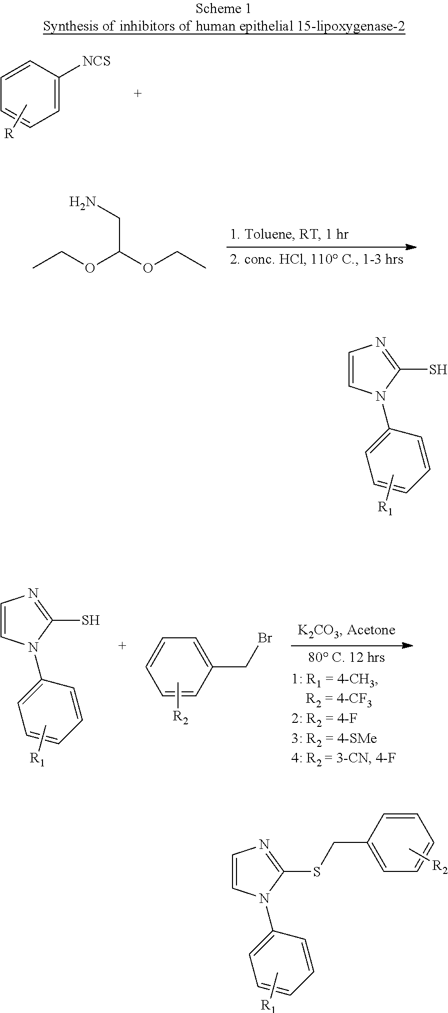

Step 1 A mixture of required phenyl isothiocyanate (5 mmol) and aminoacetaldehyde diethyl acetal (5 mmol) in toluene (10 mL) was stirred at room temperature for 1 hr. To the reaction mixture, conc. HCl (37 wt. % in water, 2.5 mmol) was added, followed by heating to reflux (bath temp. =110° C.) for 1-3 hrs. After evaporating the solvents, the residue was treated with water and 1N NaOH (the pH was set to 8). The precipitates were collected by filtration, washed with water and hexane/ether, and dried in a vacuum to give the desired imidazole-2-thiol products, which were recrystallized from an appropriate solvent (acetonitrile, methanol, or benzene) (yield: 43-53%).

Step 2 Imidazole-2-thiol product from step 1 (5 mmol) in acetone was placed in the flask and potassium carbonate (15 mmol) was slowly added. The reaction was stirred under nitrogen for 1 hr and then benzyl bromide (7.5 mmol) in ethanol (10 mL) was added. The reaction was heated to 80° C. for 12 hrs. After removal of the solvent, the residue was purified by normal phase silica chromatography (DCM/MeOH=4/1) to get the final product. (yield: 60-69%)

### 1-(p-tolyl)-2-((4-(trifluoromethyl)benzyl)thio)-1H-imidazole (Compound 101)

¹H NMR (500 MHz, CD3OD) δ 7.48-7.46 (m, 2H), 7.26-7.19 (m, 6H), 6.94-6.92 (m, 2H), 4.13 (s, 2H), 2.39 (s, 3H). ¹³C NMR (500 MHz, CD3OD) δ 143.80, 141.60, 140.19, 135.86, 130.82, 130.69, 130.47, 130.44, 126.94, 126.83, 126.49, 126.46, 126.43, 126.40, 125.04, 124.67, 40.07, 21.23. HIRMS: m/z (M+H)⁺=calculated for C₁₈H₁₅F₃N₂S, 348.0908; found, 348.0909.

### 2-((4-fluorobenzyl)thio)-1-phenyl-1H-imidazole (Compound 102)

¹H NMR (500 MHz, CDCl₃) δ 7.39-7.37 (m, 3H), 7.19-7.12 (m, 6H), 6.89-6.86 (m, 2H), 4.23 (s, 2H). ¹³C NMR (500 MHz, CDCl₃) δ 161.33, 141.64, 137.23, 133.19, 133.16, 130.78, 130.72, 129.74, 129.40, 128.63, 125.77, 122.68, 115.59, 115.42, 38.17. HIRMS: m/z (M+H)+=calculated for C₁₆H₁₃FN₂S, 284.0783; found, 284.0780.

### 2-((4-(methylthio)benzyl)thio)-1-phenyl-1H-imidazole (Compound 103)

¹H NMR (500 MHz, CDCl₃) δ 7.55-7.51 (m, 4H), 7.40-7.33 (m, 4H), 7.30-7.27 (m, 3H), 4.37 (s, 2H), 2.58 (s, 3H). ¹³C NMR (500 MHz, CDCl₃) δ 141.73, 137.77, 137.27, 134.15, 129.82, 129.56, 129.31, 128.80, 128.50, 126.80, 125.77, 122.62, 121.73, 121.69, 38.59, 16.04. HRMS: m/z (M+H)+=calculated for C₁₇H₁₆N₂S₂, 312.0755; found, 312.0753.

### 2-fluoro-5-(((1-phenyl-1H-imidazol-2-yl)thio)methyl)benzonitrile (Compound 104)

¹H NMR (500 MHz, CDCl₃) δ 7.45-7.40 (m, 5H), 7.17-7.14 (m, 3H), 7.09-7.01 (m, 2H), 4.20 (s, 2H). ¹³C NMR (500 MHz, CDCl₃) δ 161.38, 140.49, 136.98, 135.78, 135.71, 135.31, 135.28, 133.83, 130.06, 129.52, 128.83, 125.59, 122.98, 116.61, 116.45, 113.82, 37.01. HRMS: m/z (M +H)+=calculated for C₁₇H₁₂FN₃S, 309.0736; found, 309.0737.

### Protein Expression

All the LOX isozymes used were expressed and purified (h5-LOX, h12-LOX, hl5-LOX-1 and h15-LOX-2). Human leukocyte 5-lipoxygenase was expressed as a non-tagged protein and used as a crude ammonium sulfate precipitated protein. The remaining enzymes: hl5-LOX-1, h15-LOX-2 and h12-LOX were expressed as N-terminal His6-tagged proteins and purified via immobilized metal affinity chromatography (IMAC) using Ni-NTA resin. The purity of each protein was analyzed by SDS-PAGE and found to be greater than 90%.

### Lipoxygenase UV-Vis-based IC₅₀ Assay

The initial one-point inhibition percentages were determined by following the formation of the conjugated diene product at 234 nm (ε=25,000 M⁻¹ cm⁻¹) with a Perkin-Elmer Lambda 40 UV/V is spectrophotometer at 25 μM inhibitor concentration. All inhibitors that showed greater than 70% inhibition were investigated further to determine their IC₅₀ values. The full IC₅₀ experiments were done with at least five different inhibitor concentrations. All reaction mixtures were 2 mL in volume and constantly stirred using a magnetic stir bar at room temperature (23° C.) with the appropriate amount of LOX isozyme [h5-LOX (˜200 nM); h12-LOX (˜50 nM); h15-LOX-1 (˜60 nM); h15-LOX-2 (˜200 nM)]. The protein concentrations are the total protein concentration; active protein concentration can be significantly less due to incomplete metalation. Reactions with h12-LOX were carried out in 25 mM HEPES (pH 8.0) 0.01% Triton X-100 and 10 μM AA. Reactions with the crude, ammonium sulfate precipitated h5-LOX were carried out in 25 mM HEPES (pH 7.3), 0.3 mM CaCl₂), 0.1 mM EDTA, 0.2 mM ATP, 0.01% Triton X100 and 10 μM AA. Reactions with h15-LOX-1 and h15-LOX-2 were carried out in 25 mM HEPES buffer (pH 7.5), 0.01% Triton X-100 and 10 μM AA. The concentration of AA was quantitated by allowing the enzymatic reaction to proceed to completion in the presence of soybean 15-LOX-1 (s15-LOX-1). IC₅₀ values were obtained by determining the percent inhibition at various inhibitor concentrations and plotting against inhibitor concentration, followed by a hyperbolic saturation curve fit. The percent inhibition was calculated by comparing the enzymatic rate of the control (DMSO) to the enzymatic rate with the respective inhibitor present. The experiments used for generating the saturation curves were performed in duplicate or triplicate, depending on the quality of the data. All inhibitors were stored at −20° C. in DMSO.

Steady-State Inhibition Kinetics of h15-LOX-2

The steady-state equilibrium constants of dissociation for Compound 105, Compound 106 and Compound 107 were determined by monitoring the formation of the conjugated product, 15-HpETE, at 234 nm (ε=25,000 M⁻¹cm⁻¹) with a Perkin Elmer Lambda 40 UV/Vis spectrophotometer. Reactions were initiated by adding h15-LOX-2 to a constantly stirring 2 mL reaction mixture containing 0.7 μM-20 μM AA in 25 mM HEPES buffer (pH 7.5), in the presence of 0.01% Triton X-100. Kinetic data were obtained by recording initial enzymatic rates, at varied inhibitor concentrations, and subsequently fitting the data to the Henri-Michaelis-Menten equation using KaleidaGraph (Synergy) to determine Vₘₐₓ (μmol/min) and Kₘ (μM). The primary data were then plotted in Dixon format using Microsoft Excel by graphing 1/v vs. [I] μM at the chosen substrate concentrations. From the Dixon plots, the slope at each substrate concentration was extracted and plotted against 1/[S] μM to produce the Dixon replots. The K_{ic} equilibrium constant of dissociation was calculated by dividing Kₘ/Vₘₐₓ by the slope of the replot. To obtain Kᵢᵤ, 1/Vₘₐₓ was divided by the y-intercept of the replot. K_{ic} and Kᵢᵤ are defined as the equilibrium constant of dissociation from the catalytic and secondary sites, respectively.

### Pseudo-peroxidase Assay

The pseudo-peroxidase activity of Compound 105, Compound 106 and Compound 107 were determined with h15-LOX-2 on a Perkin-Elmer Lambda 40 UV/Vis spectrophotometer as described previously. 13-HpODE was used as the oxidant and BWb70c as the positive control. The reaction was initiated by addition of 20 μM 13-HpODE to 2 mL buffer (50 mM sodium phosphate (pH 7.4), 0.3 mM CaCl₂), 0.1 mM EDTA, 0.01% Triton X-100) containing 20 μM Compound 105, Compound 106 and Compound 107 and 200 nM h15-LOX-2. The reaction mixtures were constantly stirred at 23° C. The activity was determined by monitoring the decrease at 234 nm (product degradation) and the percent consumption of 13-HpODE was recorded. More than 25% 13-HpODE degradation indicates redox activity of that particular inhibitor. The negative controls used were: enzyme alone with the product, enzyme alone with inhibitor, as well as inhibitor alone with the product. These formed a baseline for the assay, reflecting non-pseudo-peroxidase dependent hydroperoxide product decomposition. To rule out the auto-inactivation of the enzyme from pseudo-peroxidase cycling, the h15-LOX-2 residual activity was determined by the addition of 20 μM AA at the end of each reaction. The initial rates of the inhibitor and 13-HpODE were compared to the initial rates of inhibitor alone because the inhibitor by itself inherently lowers the rate of oxygenation. Activity is characterized by direct measurement of the product formation with the increase of absorbance at 234 nm.

### Cyclooxygenase Selectivity Assay

Cyclooxygenase selectivity assay was performed as previously described.²⁴ Approximately 3 μg of either ovine COX-1 (COX-1) or human recombinant COX-2 (COX-2) (Cayman Chemical) were added to buffer containing 0.1 M Tris-HCl buffer (pH 8.0), 5 mM EDTA, 2 mM phenol, and 1 μM hemin at 37° C. Compound 105, Compound 106 and Compound 107 were added to the reaction cell, followed by a 5 minute incubation with either of the COX isozymes. The reaction was then initiated by adding approximately 100 μM AA in the reaction cell, as indicated in the enzymatic protocol (Cayman Chemicals). A Hansatech DWI oxygen electrode was utilized for data collection and the consumption of oxygen was recorded.

Indomethacin and the vehicle of inhibitor (DMSO) were the positive and negative controls, respectively. The percent inhibition of the enzyme was calculated by comparing the rates of O₂ consumption for experimental samples (with inhibitor) to the rates of control samples (with DMSO).

Inhibitors of human epithelial 15-lipoxygenase-2 (h15-LOX-2) as Substrates

Compound 105, Compound 106 and Compound 107 were reacted with h15-LOX-2 to determine if they act as substrates. All buffer conditions and the determination of each rate are identical to the UV-Vis assay as described above. 20 μM of each inhibitor was reacted with h15-LOX-2 in 2 mL reaction mixtures in the absence of AA. Controls included DMSO (vehicle), 10 μM AA, and enzyme. All reactions were conducted on a Perkin Elmer Lambda 40 UV/Vis spectrophotometer. No change of absorbance at 234 nm or 280 nm was observed for each reaction. Each reaction mixture was subsequently extracted and analyzed via RP-HPLC using a Higgins HAIsiL analytical column. Solution A was 99.9% ACN and 0.1% acetic acid; solution B was 99.9% H₂O and 0.1% acetic acid. An isocratic elution of 55% A:45% B was used in the HPLC analysis. Retention times and absorbance spectra of each of the reactions were compared to spectra of the controls. Collectively, the data from the UV-Vis experiments as well as the HPLC analysis confirm that these inhibitors do not act as substrates to h15-LOX-2.

Virtual Screening of Novel h15-LOX-2 Inhibitors

Virtual screening software, Glide (version 8.7, Schrodinger Suite 2020 release 2), was used to predict the binding modes of Compound 105, Compound 106 and Compound 107. The structure of h15-LOX2 co-crystallized with a substrate mimic inhibitor (hydroxyethyloxy-tri(ethyloxy)octane) was used in Glide docking (pdb id: 4NRE). Prior to docking, the protein structure was subjected to a protein-preparation step (Schrodinger Inc). During this step, appropriate bond-orders and atom types were set, hydrogen atoms were added, protonation states of titratable residues such as His, Asp and Glu were adjusted, side chains of Asn, Gln, Thr and Tyr residues were optimized to make hydrogen bond interactions and, finally, a short minimization of the whole protein structure was performed such that the heavy atoms did not move beyond 0.3 A from their starting positions. During the protein preparation step, the co-crystallized ligand, the metal ion (Fe²⁺) and a water molecule that coordinates to the metal ion were retained. The structures of the ligands identified in this study were built using Edit/Built panel of Maestro software (version 12.4, Schrodinger Inc). They were subsequently energy minimized using LigPrep software (Schrodinger Inc). The docking process consisted of grid preparation and ligand-docking steps. After the protein-preparation step, the co-crystallized ligand were removed from protein-ligand complex structure and used its coordinates to define the docking-grid box center. Inhibitors were docked using the standard-precision (SP) docking scoring function. Initial attempts to dock the inhibitors using flexible-ligand rigid-receptor docking protocol, the docking program failed to identify docking poses free of steric clashes for all three inhibitors. Therefore, both inhibitor and receptor were treated flexibly by means of InducedFit docking after opening the active site using the InducedFit docking (InducedFit Dock, Schrodinger Inc). During the InducedFit docking all residues in the active site, other than the Fe³⁺, the water molecule, and the metal-coordinating His373, His378 and His553, were treated flexibly. The protein model from the top ranking InducedFit docking pose and then docked all inhibitors using standard rigid-receptor flexible ligand docking (Glide) with the standard-precision (SP) scoring function.

Co-Structure of h15-LOX-2 and Inhibitor

The loop mutant (LM) of h15-LOX-2 has amino acids 73-79 deleted (PPVLPLL) and was previously cloned. Briefly, 15-LOX-2 LM is overexpressed in Rosetta 2 (DE3) cells in the pET Duet-1 vector with the E. col yijgD gene after promoter 2. For the catalytically inactive Mn²⁺-substituted h15-LOX-2, the bacterial culture was grown in M9 minimal media containing 0.4% (w/v) glucose, 1 mM MgSO₄, 0.1 mM CaCl₂), 100 μgmL⁻¹ thiamine, 150 μM Mn(II)(SO₄)₂, and 0.2% (w/v) casamino acids. Enzyme was purified with a 5 mL Co²⁺-HisTrap HP columns installed on an AKTA FPLC (Cytiva, formerly GE Healthcare Life Sciences). After the protein is bound, the column is washed with 20 column volumes (CV) of buffer A (20 mM Tris, 500 mM NaCl, 20 mM imidazole, pH 8.0) and eluted with a 20 CV gradient with buffer B (20 mM Tris, 500 mM NaCl, 200 mM imidazole, pH 8.0). Fractions are concentrated in Amicon Ultra-15 centrifugal filter units with a 30 kDa cutoff. Protein is applied to a Superdex 200 10/300 GL column; monomer and dimer peaks are collected and used separately for crystallization studies.

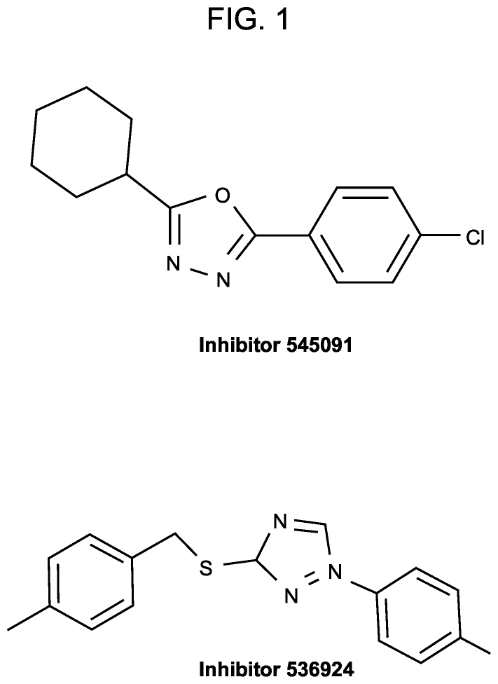

h15-LOX-2 LM Mn²⁺-substituted at 10 mg/mL with a cocktail of 500 μM of both inhibitors 545091 and 536924 (FIG. 1) was screened with sparse matrix screens from Hampton Research, Rigaku Reagents, Qiagen, and Molecular Dimensions on a Gryphon liquid dispenser (ARI).

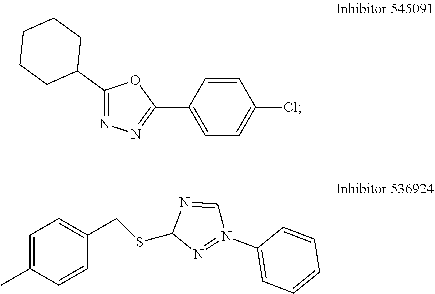

The condition of 20% Jeffamine M-2070 and 20% DMSO resulted in rod-like crystals directly from the HTS conditions. Attempts at repeating conditions by hand were unsuccessful. Jeffamine M-2070 is an industrial-grade reagent that was sold by Molecular Dimensions.

The h15-LOX-2 LM crystals were directly looped from the HTS condition and plunged into liquid N₂ for shipping. X-ray data were collected at the 24ID-E beamline of the Northeastern Collaborative Access Team at the Advanced Photon Source (Argonne National Laboratory). XDS, pointless, and Scala were used via the RAPD processing suite of the Northeastern Collaborative Access team. RAPD applies a resolution cutoff at CC_{1/2}>0.35.²⁷ Molecular replacement with h15-LOX-2 (4NRE) was performed in the Phenix program suite and two molecules were placed in the asymmetric unit. Phenix.refine and coot were used for refinement and manual model building. Phenix.elbow was used to generate restraints for the small molecule inhibitors. Density consistent with an inhibitor is present in the active site of both protomers in the asymmetric unit. Given the similarity of the structures of the two compounds, it is not possible to determine if there is a mixture of occupancy for the inhibitors or a single inhibitor, and which orientation each inhibitor binds in the active sites. Final refinements for each inhibitor positioned in the two possible binding modes in the electron density for each chain are included in the table below. The electron density clearly suggests that the inhibitor does not occupy a position in the metal coordination sphere. A water molecule mediates an interaction in chain A between the ligand and the metal. Real-space correlation coefficients and occupancies are provided in the final parallel refinements for both inhibitors in each chain and each orientation

### (Table 1).

TABLE 1

| | Real-space correlation | |
|---|---|---|
| Ligand | coefficients | Occupancy |
| Inhibitor 536924 | (chain A) 0.89 (chain B) 0.89 | (chain A) 0.65 (chain B) 0.70 |
| Inhibitor 536924 | (chain A) 0.83 (chain B) 0.89 | (chain A) 0.68 (chain B) 0.75 |
| (flipped) | | |
| Inhibitor 545091 | (chain A) 0.79 (chain B) 0.82 | (chain A) 0.68 (chain B) 0.73 |
| Inhibitor 545091 | (chain A) 0.75 (chain B) 0.78 | (chain A) 0.66 (chain B) 0.70 |
| (flipped) | | |

Hydrogen/Deuterium Exchange-Mass Spectrometry of h15-LOX-2

Aliquots of h15-LOX-2, purified from E. co/i cultures were thawed and were diluted 10-fold (5 μL into 45 μL) in 10 mM HEPES, 150 mM NaCl, pD 7.4 D₂O (99% D, Cambridge Isotopes) buffer (corrected; pD=pHread+0.4). Samples were incubated randomly at 10 time points (0, 10, 20, 45, 60, 180, 600, 1800, 3600, and 7200 s) at 25° C. using a water bath. For each given temperature and mutant, the time points (samples) were collected over the course of three to four days and randomized to reduce systematic error. Each sample (from a unique time point) was prepared and processed once. For the samples containing inhibitors, the inhibitor was added (20 μM final concentration) to the protein stock solution (at least one minute) prior to D₂O addition. The specific inhibitor was also added to the D₂O buffer at a final concentration of 20 μM prior to the exchange experiment.

Upon completion of the designated incubation time, all samples were then treated identically; the samples were rapidly cooled (5-6 seconds in a −20° C. bath) and acid quenched (to pH 2.4, confirmed with pH electrode, with 0.32 M citric acid stock solution [90 mM final concentration] at 0° C.). Procedures from this point were conducted near 4° C. Prior to pepsin digestion, guanidine HCl (in citric acid, pH 2.4) was mixed with the samples to a final concentration of ca. 0.5 M. This solution contained DTT to the final concentration of 5 mM. The addition of the reducing and chaotropic agents were necessary for obtaining high coverage of the primary sequence (90-94%). 15-LOX-2 samples were digested with pre-equilibrated (10 mM citrate buffer, pH 2.4), immobilized pepsin for 2.5 min. The peptide fragments were filtered, removing the pepsin, using spin cups (cellulose acetate) and by centrifugation for 10 seconds at 4° C. Samples were flash-frozen immediately in liquid nitrogen and stored at −80° C. until data collection.

Deuterated, pepsin-digested samples of 15-LOX-2 were analyzed using an Agilent 1200 LC (Santa Clara, CA) that was connected in-line with the LTQ Orbitrap XL mass spectrometer (Thermo). Mass spectral data acquired for HDX measurements were analyzed using the software, HDX WorkBench. The percent deuterium incorporation was calculated for each of these peptides, taking into account the number of amide linkages (excluding proline residues) and the calculated number of deuterons incorporated. The values were normalized for 100% D₂O and corrected for peptide-specific back-exchange, HDX %=(observed, normalized extent of deuterium incorporation {in percent})/(1-{BE/100}). Back-exchange values ranged from 17 to 60%, for an average value of 36%. The resulting data were plotted as deuterium exchange versus time using Igor Pro software.

Inhibition of h15-LOX-2 in HEK293T cells

HEK293T cells over-expressing h15-LOX-2 were grown in MEM (Gibco) with 10% FBS (Gibco), 2 mM glutamine (Sigma), 100U/mL of penicillin/streptomycin (Gibco) and 640 μg/ml G418 sulfate (Fisher) as a selection agent. Cells were harvested at 90% confluence with trypsin-EDTA (Gibco) and washed once with MEM with 10% FBS. Cells were then washed with 0.1% glucose (Fisher) in PBS (Gibco). Cells were then diluted in 0.1% glucose in PBS to a concentration of 1 million cells/mL. Cells were treated with DMSO (0.2%) or inhibitor in DMSO and incubated at 37° C. for 20 min. Cells were then stimulated with 100 uM CaCl₂) (Sigma), 5 μM Ca Ionophore A23187 (Sigma), and 1 μM arachidonic acid (NuCheck) for 10 min at 37° C. Cells were then acidified to 40 uM HCl and snap-frozen in liquid nitrogen. Analysis of 15-HETE was 15 performed with the addition that MS/MS m/z transition 319.2-4219 was used to measure 15-HETE. Leftover cells from the inhibition assay were used to seed flasks and grew as well as untreated cells. Additionally, a cell survival assay was performed, in which cells were treated with 0.2% DMSO, 10 μM inhibitor, or 20 μM inhibitor for hr. After incubation, the media was replaced, and the cells were monitored daily to assess cell death.

### Results and Discussion

### Compound Identification and Inhibitor Potency

IC₅₀ values for inhibitors Compound 105, Compound 106 and Compound 107 targeting h15-LOX-2 with error in parentheses are summarized in Table 2. All experiments were conducted in duplicate and with 10 μM AA. The IC₅₀ values for inhibitors 545091 and 536924 are also provided for comparison.

TABLE 2

| Inhibitor | Structure | IC₅₀ (μM) (± SD (μM)) |
|---|---|---|
| Inhibitor 545091 | 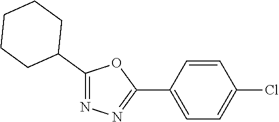 [SMILES: Clc1ccc(-c2nnc(C3CCCCC3)o2)cc1] | 2.6 (0.4) |
| Inhibitor 536924 | 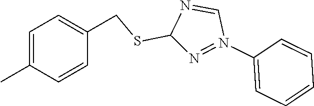 | 3.1 (0.4) |
| Compound 105 | 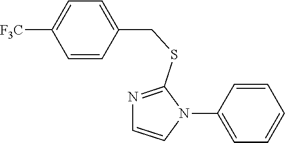 [SMILES: FC(F)(F)c1ccc(CSc2nccn2-c2ccccc2)cc1] | 0.34 (0.05) |
| Compound 106 | 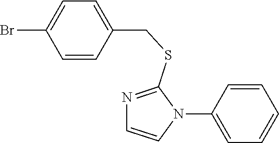 [SMILES: Brc1ccc(CSc2nccn2-c2ccccc2)cc1] | 0.53 (0.04) |
| Compound 107 | 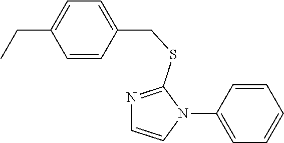 [SMILES: CCc1ccc(CSc2nccn2-c2ccccc2)cc1] | 0.87 (0.06) |

In the in vitro LOX assay, Compound 105 inhibited hl5-LOX-2 with an IC₅₀ of 0.34+0.05 μM, Compound 106 had a potency of 0.53+0.04 μM and Compound 107 showed a similar potency of 0.87+0.06 μM.

### Steady-State Inhibition Kinetics

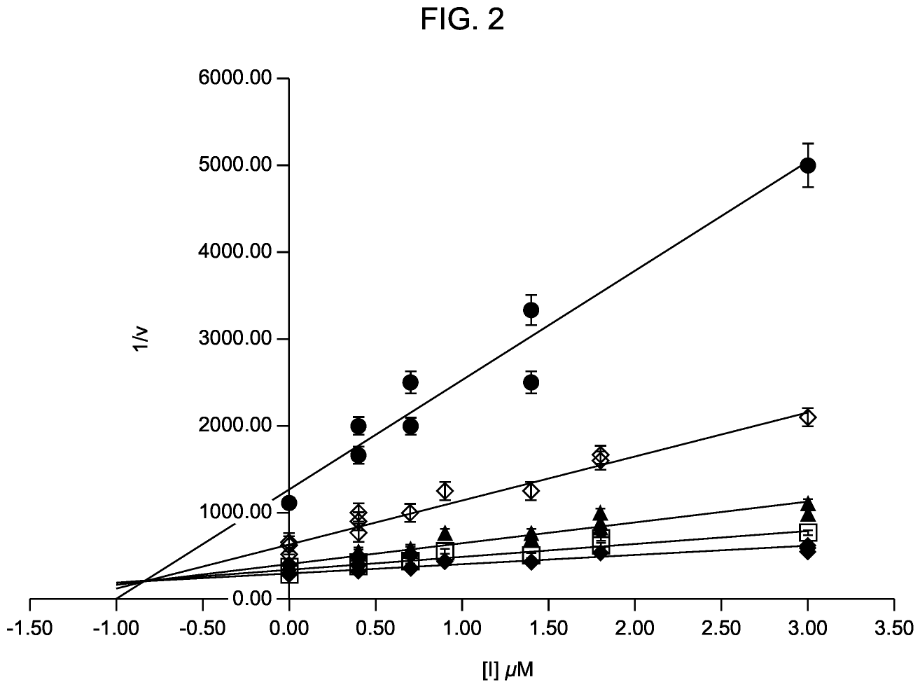

Given that Compound 105, Compound 106 and Compound 107 exhibited potency against h15-LOX-2, their modes of inhibition were probed utilizing steady-state inhibition kinetics. The formation of 15-HpETE was monitored as a function of substrate and inhibitor concentration in the presence of 0.01% Triton-X-100. Fitting the data for Compound 106 yielded mixed-type inhibition with a K_{ic} of 0.80+0.05 μM and a Kᵢᵤ of 4.0±3 μM, which are defined as the equilibrium constants of inhibitor dissociation from the enzyme and enzyme-substrate complex, respectively (FIG. 2). The steady-state inhibition kinetic experiments were also performed for Compound 105 and Compound 107 (Table 3), with the inhibition constants of all three molecules being comparable, as determined from their Dixon plots and Dixon replots. The data demonstrate that all three inhibitors exhibit mixed inhibition against h15-LOX-2, with the trends in the inhibition equilibrium constants being consistent with the IC₅₀ values (Table 2). Table 3 summarizes the equilibrium constant of dissociation from the catalytic (K_{ic}) and secondary (Kᵢᵤ) sites extracted from Dixon plots and Dixon replots of h15-LOX-2 and inhibitor 545091, inhibitor 536924, Compound 105, Compound 106 and Compound 107. Inhibitor 536924 displayed competitive inhibition.

TABLE 3

| | | |
|---|---|---|
| Inhibitor | K_{ic} (μM) | Kᵢᵤ (μM) |

| 545091 | 0.90 | (0.4) | 9.9 | (0.7) |
|---|---|---|---|---|
| 536924 | 2.5 | (0.5)^{b} | N/A | |
| Compound 105 | 0.70 | (0.07) | 1.2 | (0.9) |
| Compound 106 | 0.80 | (0.05) | 4.0 | (3) |
| Compound 107 | 0.90 | (0.04) | 3.4 | (2) |

### Selectivity Assays

Once the potencies of Compound 105, Compound 106 and Compound 107 against h15-LOX-2 had been determined, their selectivity against h5-LOX, h15-LOX-1, h12-LOX, COX-1, and COX-2 was investigated. All three molecules displayed high selectivity, at least 50-fold, for h15-LOX-2 against all enzymes tested (Table 4). Table 4 summarizes the full IC₅₀ experiments were performed with h15-LOX-2, while for the other oxygenases the IC₅₀ values were estimated at 25 μM inhibitor concentration. Inhibition that was less than 15% and less than 30% at 25 μM are estimated to have IC₅₀ values of >100 μM and >50 μM, respectively. All experiments were done in duplicate, and all assays were performed with 10 μM AA except for the cyclooxygenases which were conducted at 100 μM AA. The values are in units of micromolar with error displayed in the parentheses.

TABLE 4

| Inhibitors | h15-LOX-2 | h15-hLO-1 | h12-LOX | h5-LOX | COX-1 | COX-2 | Redox |
|---|---|---|---|---|---|---|---|
| Compound 105 | 0.34 (0.05) | >50 | >100 | >100 | >100 | >100 | No |
| Compound 106 | 0.53 (0.04) | >50 | >100 | >100 | >100 | >100 | No |
| Compound 107 | 0.87 (0.06) | >50 | >100 | >100 | >100 | >100 | No |

### Pseudo-Peroxidase Activity Assay

To better understand the mechanism of inhibition between Compound 105, Compound 106 and Compound 107 and h15-LOX-2, the redox capability of the three molecules was investigated in a pseudo-peroxidase activity assay. Although many LOX inhibitors in the literature exhibit redox activity, they are not regarded as good therapeutics due to their tendencies for off-target redox reactions. All three inhibitors were tested using the UV-Vis pseudo-peroxidase assay, with the lack of degradation of 13-HpODE at 234 nm confirming that the inhibitors are not redox-active (Table 4).

Substrate Activity of Inhibitors of human epithelial 15-lipoxygenase-2 (h15-LOX-2)

To determine whether h15-LOX-2 can catalytically modify the inhibitors, 20 μM of each inhibitor was reacted with h15-LOX-2 and the reaction monitored at 205 nm, 234 nm and 280 nm. An increase in absorbance at each wavelength was not detected indicating no chemical reaction. To confirm these results, the reactions were extracted, dried under N₂ and brought up in methanol for RP-HPLC analysis. No significant difference in spectra or retention time was observed at 212 nm (λₘₐₓ of inhibitor), 234 nm or 280 nm, confirming that Compound 105, Compound 106 and Compound 107 are not substrates to h15-LOX-2.

### Structure/Activity Relationship (SAR) Study

A SAR study was performed. (Table 5). Table 5 summarizes the h15-LOX-2 IC₅₀ values of h15-LOX-2 inhibitors for the structure/activity relationship study; errors when available are in brackets. Experiments were conducted in the presence of 10 μM AA with 0.01% TritonX-100, 20 μM inhibitor concentration and were performed in duplicate. Compound 105, Compound 106 and Compound 107 possess 3 different substituents at the para position of the benzylthio moiety, a trifluoromethyl (Compound 105), a bromo (Compound 106), and an ethyl (Compound 107) moiety, all of which demonstrated comparable potency (Table 2). To probe the structure-activity relationship further, additional analogs containing variations on the benzylthio and N-phenyl moieties were synthesized. From the aggregate data (Table 5). A clear SAR trend was not observed from the structural modifications, though most of the similar analogs showed lower or comparable potencies to the original hits. For example, Compound 103 with p-SCH3 group showed slightly decreased potency with an IC₅₀=2.3 μM. Compound 102 with a p-fluoro group showed no activity up to 100 μM. Likewise, ortho or meta substitution in the phenyl ring (Compounds 104, 108—and 109) or extending the linker length (Compound 110) drastically decreased the potency. A similar trend was observed for Compound 111-Compound 127 which have drastic structural modifications in the form of bulkier groups, amides, or acids, possibly due to disruption of the three-dimensional orientation of the molecules that can no longer fit in the binding pocket. Introducing a p-methyl to the N-phenyl ring (Compound 101), slightly improved the potency with an IC₅₀=0.27 μM. In summary, a minor SAR effect was observed and that major structural changes were not well tolerated.

TABLE 5

| | | % Inhibition | IC50 (μM) |
|---|---|---|---|
| Inhibitors | Structure | at 20 μM | (± SD) |
| Compound 105 | 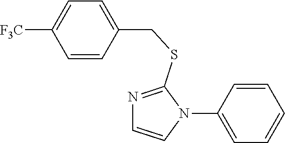 [SMILES: FC(F)(F)c1ccc(CSc2nccn2-c2ccccc2)cc1] | 100% | 0.34 (0.05) |
| Compound 106 | 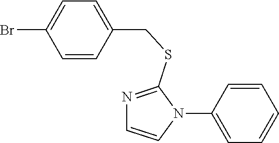 [SMILES: Brc1ccc(CSc2nccn2-c2ccccc2)cc1] | 100% | 0.53 (0.04) |
| Compound 107 | 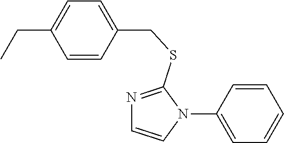 [SMILES: CCc1ccc(CSc2nccn2-c2ccccc2)cc1] | 98% | 0.87 (0.06) |
| Group 1 | | | |
| Compound 101 | 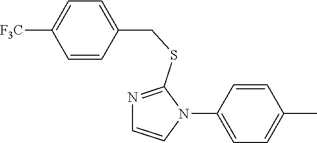 [SMILES: Cc1ccc(-n2ccnc2SCc2ccc(C(F)(F)F)cc2)cc1] | 97% | 0.27 (0.07) |
| Compound 102 | 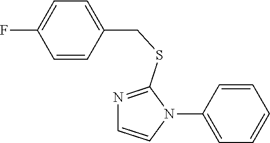 [SMILES: Fc1ccc(CSc2nccn2-c2ccccc2)cc1] | 35% | >100 |
| Compound 103 | 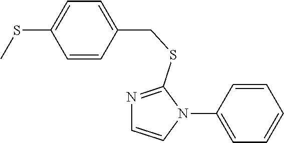 [SMILES: CSc1ccc(CSc2nccn2-c2ccccc2)cc1] | 84% | 2.3 (0.3) |
| Compound 104 | 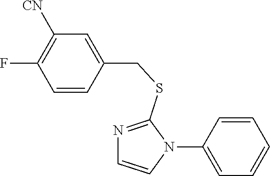 | 23% | >100 |
| Compound 108 | 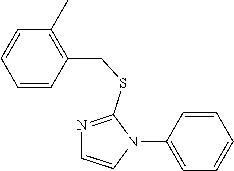 [SMILES: Cc1ccccc1CSc1nccn1-c1ccccc1] | 31% | >50 |
| Compound 109 | 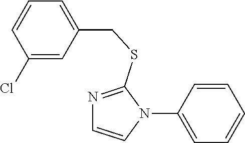 [SMILES: Clc1cccc(CSc2nccn2-c2ccccc2)c1] | 39% | >50 |
| Compound 110 | 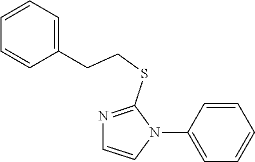 [SMILES: c1ccc(CCSc2nccn2-c2ccccc2)cc1] | 68% | >25 |
| Compound 111 | 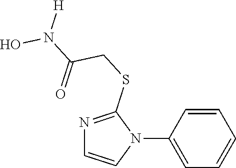 [SMILES: O=C(CSc1nccn1-c1ccccc1)NO] | 4% | >100 |
| Compound 112 | 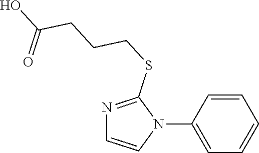 [SMILES: O=C(O)CCCSc1nccn1-c1ccccc1] | 8% | >100 |
| Compound 113 | 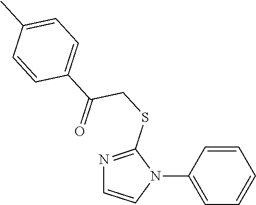 [SMILES: Cc1ccc(C(=O)CSc2nccn2-c2ccccc2)cc1] | 56% | >25 |
| Compound 114 | 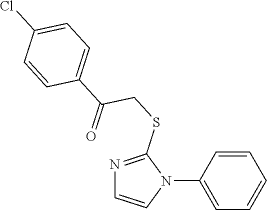 [SMILES: O=C(CSc1nccn1-c1ccccc1)c1ccc(Cl)cc1] | 62% | >25 |
| Compound 115 | 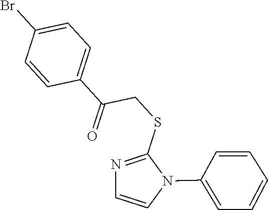 [SMILES: O=C(CSc1nccn1-c1ccccc1)c1ccc(Br)cc1] | 51% | >25 |
| Compound 116 | 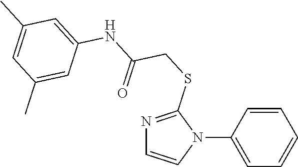 [SMILES: Cc1cc(C)cc(NC(=O)CSc2nccn2-c2ccccc2)c1] | 0% | >100 |
| Compound 117 | 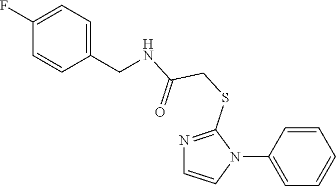 [SMILES: O=C(CSc1nccn1-c1ccccc1)NCc1ccc(F)cc1] | 8% | >100 |
| Compound 118 | 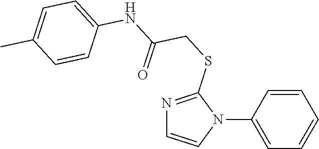 [SMILES: Cc1ccc(NC(=O)CSc2nccn2-c2ccccc2)cc1] | 18% | >50 |
| Group 2 | | | |
| Compound 119 | 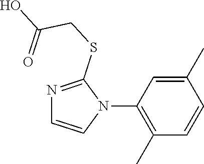 [SMILES: Cc1ccc(C)c(-n2ccnc2SCC(=O)O)c1] | 4% | >100 |
| Compound 120 | 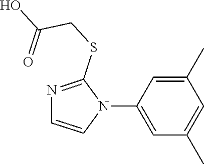 [SMILES: Cc1cc(C)cc(-n2ccnc2SCC(=O)O)c1] | 7% | >100 |
| Compound 121 | 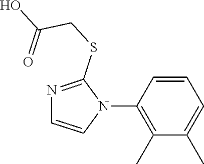 [SMILES: Cc1cccc(-n2ccnc2SCC(=O)O)c1C] | 7% | >100 |
| Compound 122 | 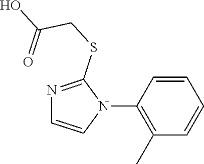 [SMILES: Cc1ccccc1-n1ccnc1SCC(=O)O] | 8% | >100 |
| Compound 123 | 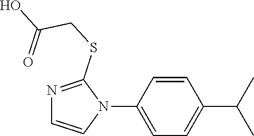 [SMILES: CC(C)c1ccc(-n2ccnc2SCC(=O)O)cc1] | 13% | >100 |
| Compound 124 | 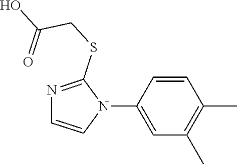 [SMILES: Cc1ccc(-n2ccnc2SCC(=O)O)cc1C] | 14% | >100 |
| Compound 125 | 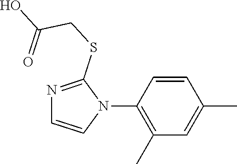 [SMILES: Cc1ccc(-n2ccnc2SCC(=O)O)c(C)c1] | 16% | >50 |
| Group 3 | | | |
| Compound 126 | 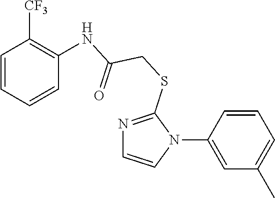 [SMILES: Cc1cccc(-n2ccnc2SCC(=O)Nc2ccccc2C(F)(F)F)c1] | 4% | >100 |
| Compound 127 | 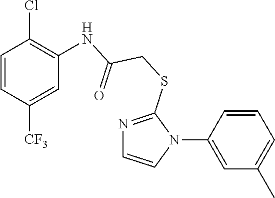 [SMILES: Cc1cccc(-n2ccnc2SCC(=O)Nc2cc(C(F)(F)F)ccc2Cl)c1] | 13% | >100 |

Computational Docking of h15-LOX-2 Inhibitors

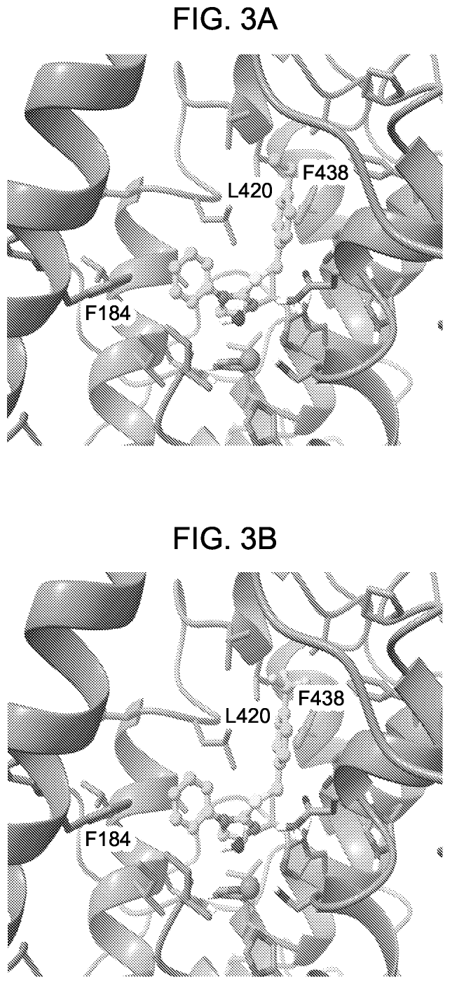

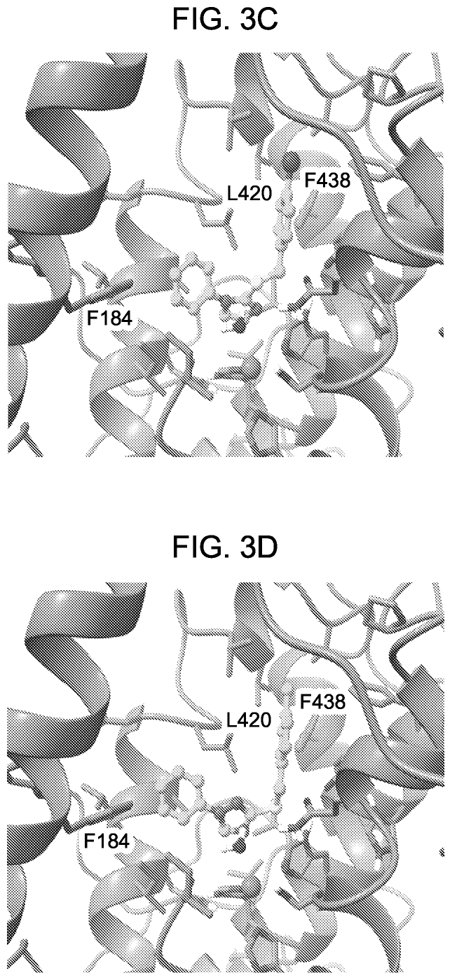

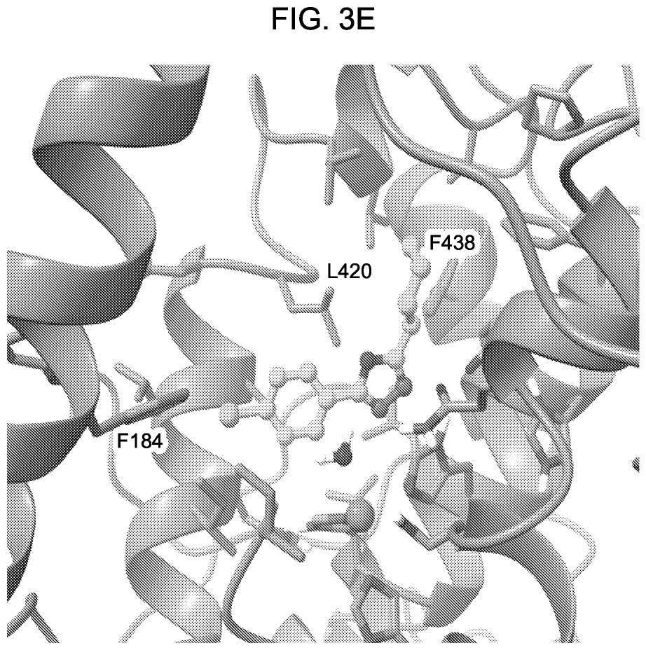

In order to predict binding modes of Compound 105, Compound 106 and Compound 107, inhibitor 545091 and inhibitor 536924, we docked them using the method described above, and docking poses are shown in FIG. 3 and Table 6. The inhibitors bind in the U-shaped active site, with the heterocyclic ring occupying the pocket near the metal ion and both aromatic rings filling the hydrophobic pockets on either side of the heterocycle. For all inhibitors, except inhibitor 536924 and inhibitor 545091, the central heterocyclic ring makes a pi-stacking interaction with the side chain of His373 (FIG. 3). The binding affinities predicted by the docking score (Glide SP) or MM-GBSA energy (Table 6) correlate weakly with the narrow range of experimental IC₅₀ values, with the inhibitors Compound 105, Compound 106 and Compound 107 ranking better than inhibitor 545091 and inhibitor 536924. These docking poses demonstrate that the heterocyclic ring and the positions of the substituents attached to the heterocyclic ring are different among these compounds. Substituents attached at these carbon and nitrogen atoms, as found in Compound 105, Compound 106 and Compound 107, might facilitate the favorable pi-stacking interaction for these compounds with F438.

TABLE 6

| | Glide SP | MM-GBSA | |
|---|---|---|---|
| | Docking | Relative binding energy | h15-LOX-2 |
| Compound | Score | (kcal/mol) | IC₅₀ (μM) |
| Compound 105 | −8.1 | 3.23 | 0.34 |
| Compound 106 | −7.8 | 6.83 | 0.53 |
| Compound 107 | −8.2 | 12.35 | 0.87 |
| Inhibitor 545091 | −7.0 | 29.76 | 2.6 |
| Inhibitor 536924 | −7.7 | 20.10 | 3.1 |

Co-Structure of h15-LOX-2 and Inhibitor

The crystallization condition of h15-LOX-2 required a detergent for high-resolution structure determination. The substrate-mimicking, competitive-inhibitor detergent, octyltetraethylene glycol ether (C8E4), was bound in the active site described as a U-shaped channel. An additional detergent molecule was revealed next to the catalytic domain near helix-α2 (Hα2); it mediates crystal contacts. Attempts at utilizing these conditions for co-inhibitor structural studies were fruitless due to the requirement of the C8E4 detergent. New crystallization conditions were screened for a loop mutant of 15-lipoxgenase-2 (h15-LOX-2 LM), with amino acid residues 73-79 being deleted (PPVLPLL). In the structure of the wildtype enzyme the hydrophobic loop projects from the P-barrel domain and hinders access to the active site of a neighboring promoter in the crystal lattice. Data suggest that peripheral insertion of this loop into the bilayer is the primary membrane-binding determinant for Ca²⁺-signaling and targeting. A cocktail of inhibitors inhibitor 545091 and inhibitor 536924 was used in the crystallization trials of h15-LOX-2 LM substituted with Mn²⁺. This substituted h15-LOX-2 LM with Mn²⁺ showed no kinetic activity, as despite the fact that the metal coordination is equivalent in the substituted enzymes, the enzymes are inactive. Crystals grew in our high-throughput screening (HTS) effort in conditions of 20% DMSO and 20% Jeffamine M-2070. Rod-shaped crystals directly from the HTS condition were flash-frozen and x-ray diffraction data were collected. The protein crystallized in space group C2 and data was collected to 2.4 Å resolution. Two monomers were positioned in the asymmetric unit of the search model h15-LOX-2 WT (4nre.pdb). According to the macromolecular interface tool PISA (Proteins, Interfaces, Structures and Assemblies), this interface is significant and could represent the dimer observed in size-exclusion chromatography.

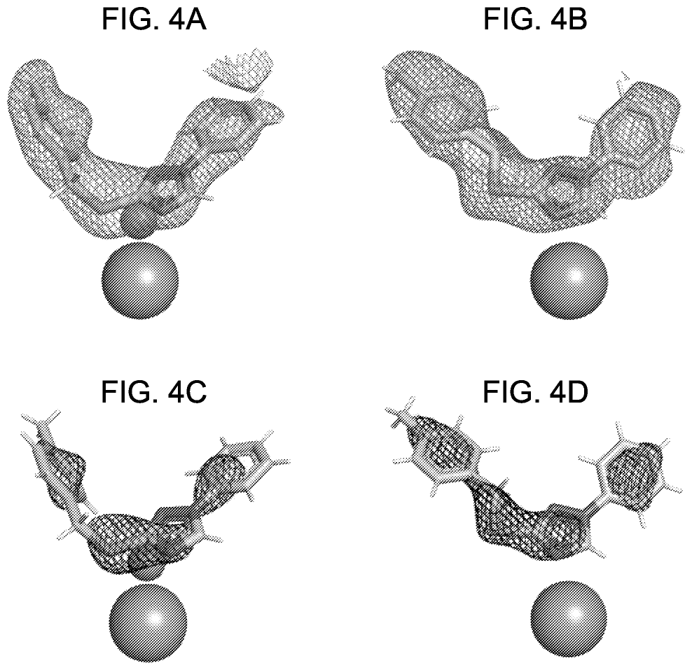

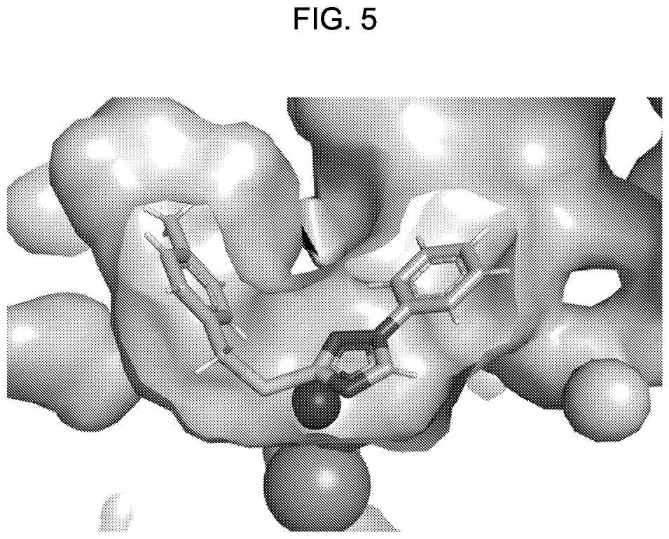

Additional electron density |Fo-Fc| near the active site metal was resolved after subsequent refinement (FIG. 4). The different inhibitors were modeled and refined in parallel refinements in the density near the active site metal. However, we are unable to unambiguously identify which inhibitor(s) is(are) present. Real-space correlation coefficients for inhibitor 536924 (0.88 CC and 0.89 CC) in both chains correlate to a higher degree versus inhibitor 545091 (0.79 CC and 0.82 CC). We can, however, unambiguously state that the inhibitors bind in the U-shaped channel and do not directly interact with the active-site metal (FIG. 5).

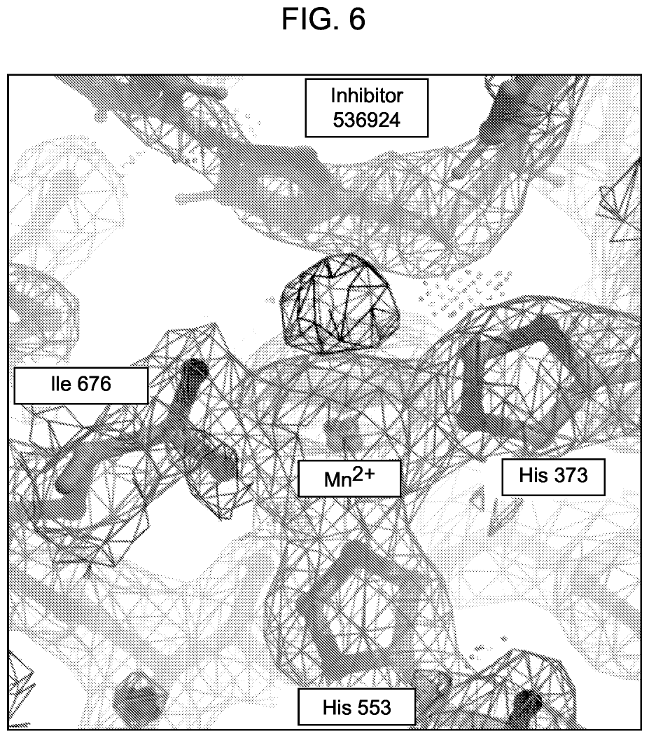

Additionally, a water molecule that occupies the open sixth position of the octahedral-coordinated Fe²⁺ in the wildtype structure was observed in chain A of the crystal structure of the Mn²⁺-substituted h15-LOX-2 LM (FIG. 6). When inhibitor 536924 is placed in the electron density near the metal, the sulfur of the inhibitor forms an H-bond with the Mn²⁺-coordinated water and His 373. All other potential interactions of inhibitors in the crystal structures share only van der Waal contacts in the U-shaped pocket defined by Ile 412, Phe 365, Thr 431, and Leu 420, which positions the targeted pentadiene of AA for hydrogen atom abstraction. The crystal structure validates many key findings of the docking experiments including water-mediated binding of the inhibitor to the metal coordination sphere near the imidazole heterocycle, and the aromatic structures of the inhibitors positioned on opposite ends of the U-shaped channel. The minimal differences of the atomic positions of amino acids in the original wild-type structure of h15-LOX-2 bound to the detergent C8E4 and our new structure of h15-LOX-2 LM co-crystallized with a cocktail of inhibitors (71af.pdb) further validate and justify the in silico rigid-body docking strategy performed.

Hydrogen/Deuterium Exchange Mass Spectrometry of h15-LOX-2

Hydrogen deuterium exchange mass spectrometry (HDX-MS) is often utilized to assess the effects of binding small molecule effectors, regulators, and inhibitors on protein conformational flexibility. Therefore, it offers an incisive high-throughput technique, complementary to the high-resolution X-ray crystal structures solved with inhibitors bound, to screen inhibitors and to resolve their impact on protein structure and flexibility. In the specific case of lipoxygenases, allosteric effectors and substrates have been shown to influence the HDX properties of both soybean lipoxygenase-1 (SLO-1) and human 15-LOX-2.

The impact of the isozyme selective inhibitor 536924 and Compound 105 were examined. Tandem MS analysis of pepsin-generated peptides of 15-LOX-2 identified 242 peptides corresponding to 94% coverage of the primary sequence. This represents a significant increase in primary sequence coverage from the previous report of 72%. For data reduction purposes, 44 non-overlapping peptides, ranging from 5 to 25 amino acids (average length, 12), were selected for HDX-MS analysis. The peptide list was well covered over all 10 time points, ranging from 10 seconds to 2 hours, and identical for samples prepared with inhibitors.

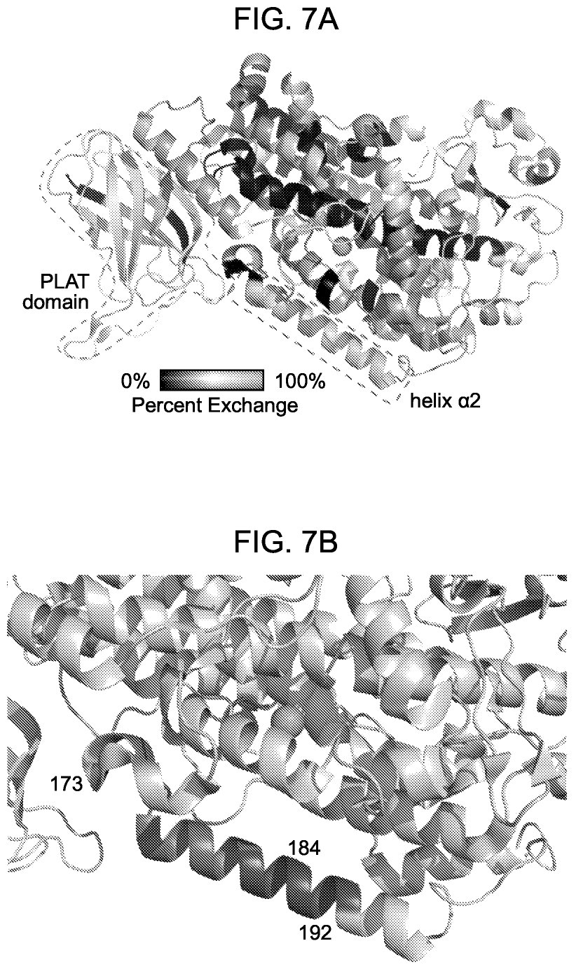

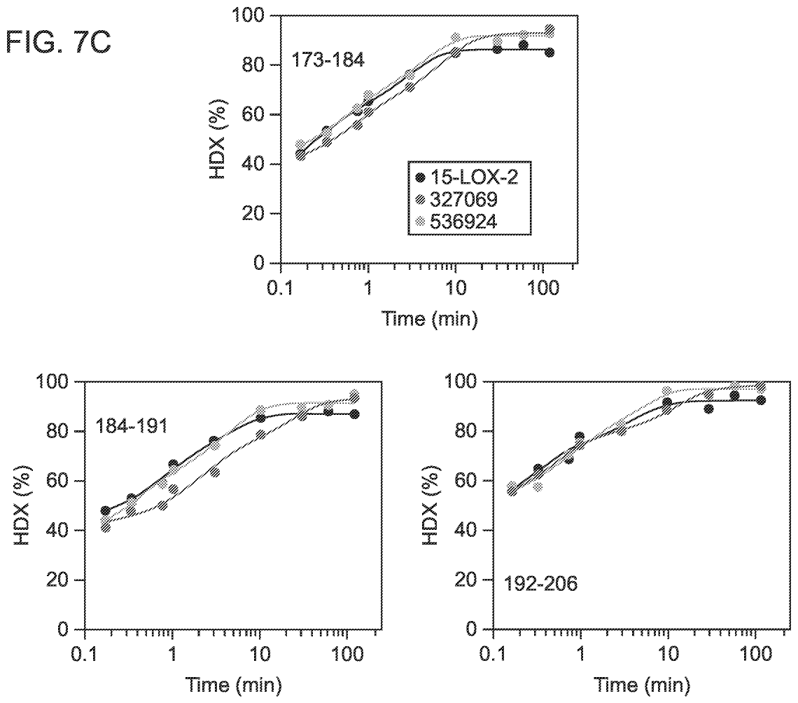

The percent exchange at 2 h for samples of h15-LOX-2 alone is mapped onto the crystal structure of 15-LOX-2 (FIG. 7A) that was solved with a substrate mimic, C8E4, in the active site. The exchange behavior of helix-α2, a peptide which restricts the substrate entrance portal and flexibility has been implicated to play an important regulatory role in substrate binding in LOXs.

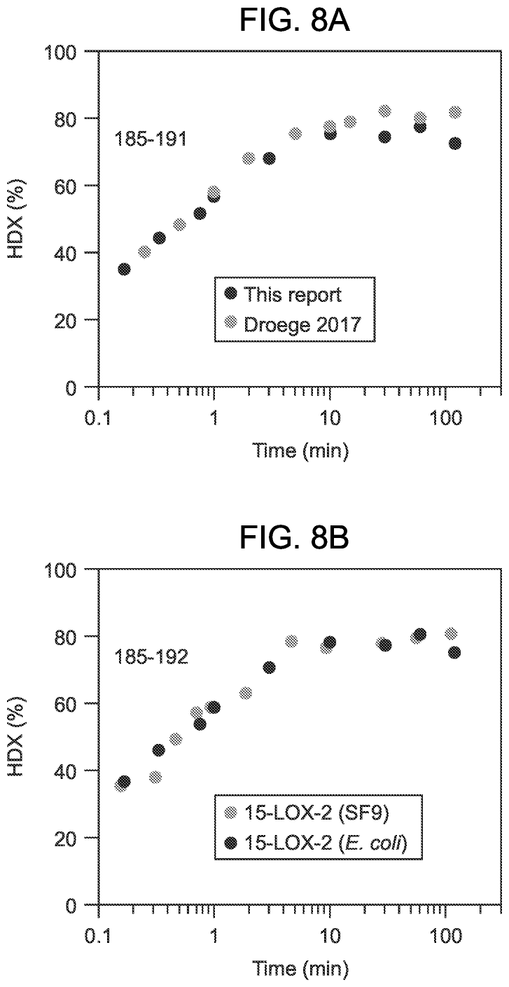

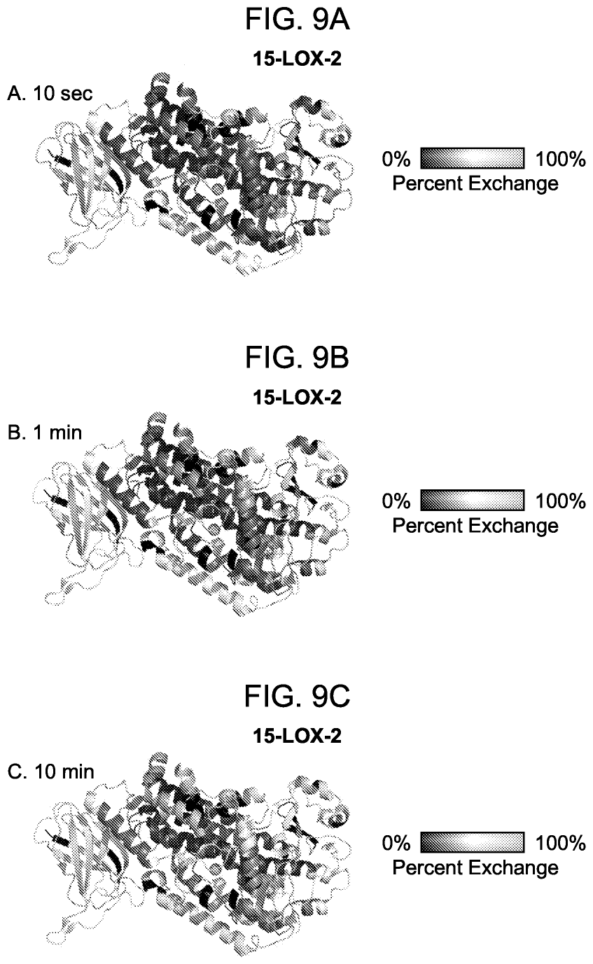

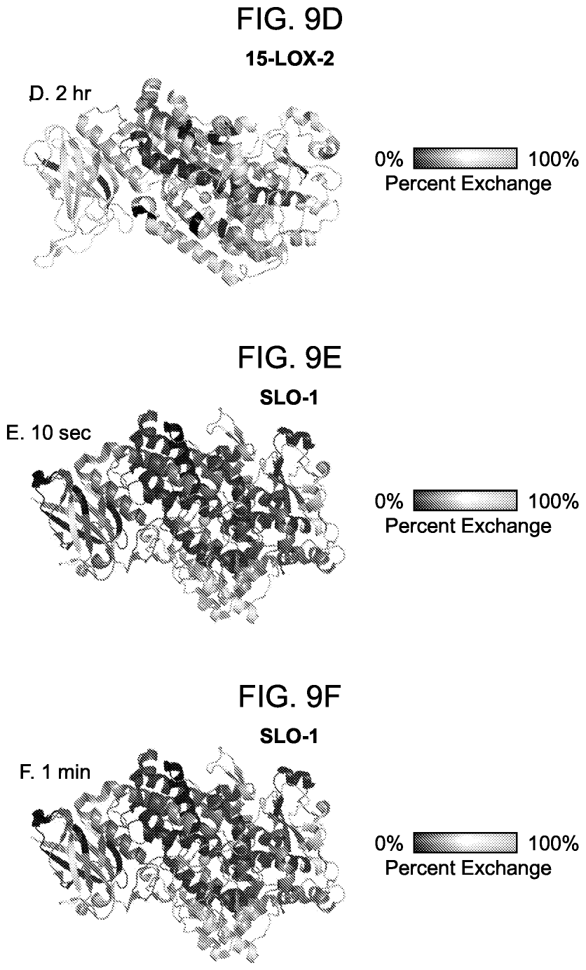

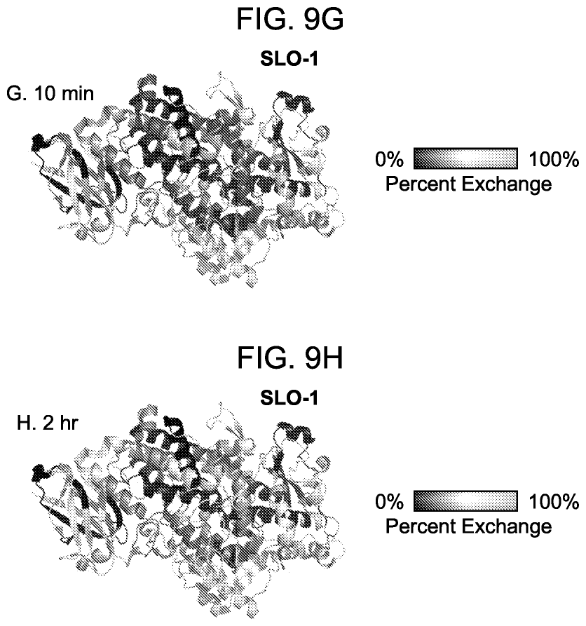

Peptide 184-191 (and its overlapping counterparts) is located in the central region of helix α2 and flanked by peptides 173-184 (FIG. 7B, chartreuse) and 192-206 (FIG. 7B, salmon). All three peptides are highly exchanged (>80%) at 2 h (FIGS. 7A and 7B). The variability in protein handling, mass spec instrumentation used, and potential differences in buffer preparation, the exchange properties of this helix α2 is nearly identical as previously reported (FIG. 8A). Further, to corroborate our h15-LOX-2 HDX properties, HDX-MS of h15-LOX-2 isolated from insect cell (SF9) cultures was conducted and compared to the HDX-MS results for h15-LOX-2 isolated from E. coli cultures. Exchange properties are nearly superimposable including helix α2 (FIG. 8B). The observed elevated exchange behavior for a crystallographically resolved alpha-helical peptide is comparable to that described for the model 15-lipoxygenase from plants, SLO-1 (FIG. 9).

One notable distinction between h15-LOX-2 and SLO-1 is the general increase in the exchange of the N-terminal PLAT domain with an average 10% higher overall extent of exchange for 15-LOX-2 (FIG. 9). In addition, the linker peptide between the PLAT and catalytic domains, 117-134, in h15-LOX-2 exhibits a significantly higher exchange (87% at 2 h) compared to the corresponding peptide in SLO-1 of ca. 45% (residues 137-160). The trend in these HDX properties of the PLAT domain is consistent with the previously reported small-angle X-ray scattering (SAXS) experiments. SAXS data of rabbit 15-LOX-1 suggested high mobility of the PLAT domain, supporting a ‘rocking’ motion that has been proposed to play a role in membrane binding. Conversely, SAXS analysis of SLO-1 indicates no significant mobility of the PLAT domain.

Impact of isozyme-selective inhibitors on h15-LOX-2 Hydrogen/Deuterium Exchange properties

The addition of substrate arachidonic acid (AA) to h15-LOX-2 was previously found to decrease the exchange behavior of peptide 185-191 (helix α2) at early time points (15-120 s) by as much as 20%. This was accompanied by a modest impact on the exchange percentage at the arched helix that runs nearly parallel to helix α2 and lines the substrate-binding site.

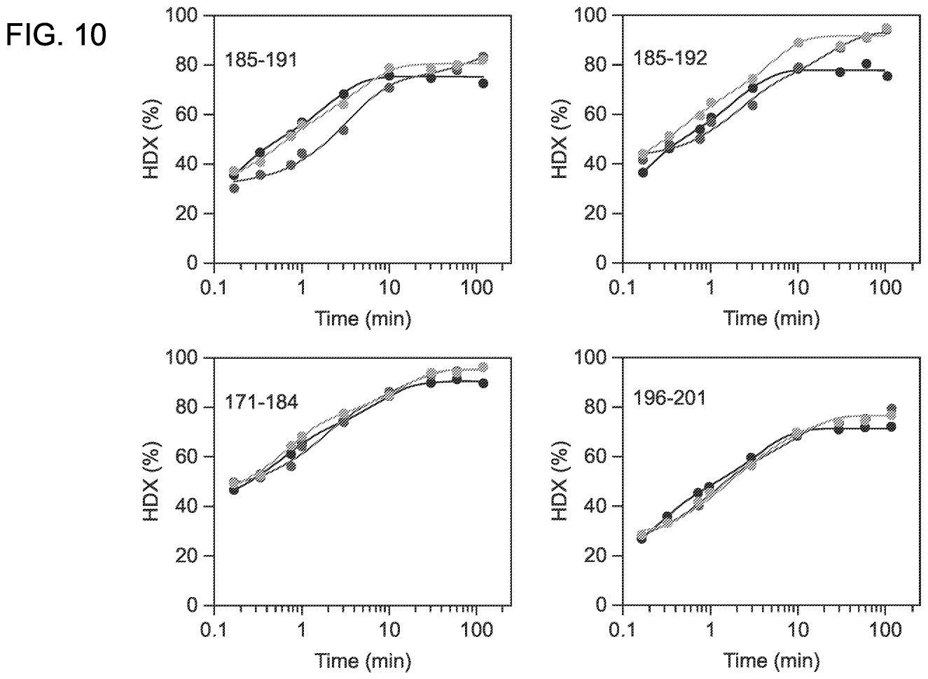

In the HDX study described herein of h15-LOX-2, two inhibitors were selected, inhibitor 536924 and Compound 105, that represented the two different chemotypes of this study. In the presence of saturating concentration (20 μM) of Compound 105, two non-overlapping hl5-LOX-2 peptides (173-184 and 184-191) were associated with a significant reduction in exchange by as much as 20% at short time scales (<10 min). From the bi-exponential fits of the time-dependent HDX traces (FIG. 7C), the average rate constants for these peptides were found to be 2—and 4-times slower in the presence of Compound 105. These trends were validated from analysis of overlapping peptides in this region (FIG. 10). This behavior is consistent with a rigidification of regional protein motions and is the canonical HDX behavior reported for protein inhibitors. The rigidification of helix α2 in the presence of Compound 105 was also similar to the HDX behavior previously characterized at short time scales for the presence of AA. Conversely, within the dynamic range of the experiment employed, HDX samples of h15-LOX-2 prepared with inhibitor 536924 showed no significant exchange differences to samples with 15-LOX-2 alone (cf FIG. 7C, black and gray traces). The difference in HDX behavior observed here for the two different h15-LOX-2 inhibitors can be attributed to the near 10-fold enhanced inhibition of Compound 105 (IC₅₀=0.34 μM) over inhibitor 536924 (IC₅₀=3.1 μM). The observation that the more potent inhibitor resulted in the structural rigidification of h15-LOX-2 helix α2 underscores the utility of HDX-MS as a powerful, high-throughput method for screening h15-LOX-2 inhibitors.

Notable decreased HDX-MS behavior for h15-LOX-2 peptides in the PLAT domain, namely 45-52, 54-66, and 67-86 with the addition of the AA substrate has been previously reported. However, from the present study even under saturating inhibitor concentrations, there is no significant effect on the apparent HDX rate in this region between the samples in the presence and absence of either inhibitor. Note that at long time points, the inhibitors cause a slight increase in the extent of exchange, including peptide 45-53. Because this is only observed at longer time points, this effect is attributed to a possible regional dependent destabilization of the protein structure in the presence of the inhibitors.

This distinction between the exchange behavior reported for AA and that described for the inhibitors described herein could be catalytically significant. Note that during the 15-120 second exchange experiment with AA, the substrate will be converted to 15-HpETE. While not fully resolved for h15-LOX-2, conformational changes are expected to accompany substrate binding, turnover, and product release. Further, AA could potentially bind at an allosteric site in h15-LOX-2 and elicit altered protein conformational ensembles. The substrate selectivity of h15-LOX-2 has been shown to be influenced by allosteric effectors, including 13S-HODE, an enzymatic product from the reaction of hl5-LOX-1 with linoleic acid. Allosteric effects in the HDX behavior have also been detected in the PLAT domain of SLO-1 when using the SLO-1 allosteric effector, oleyl sulfate.

Inhibition of h15-LOX-2 in HEK293T cells

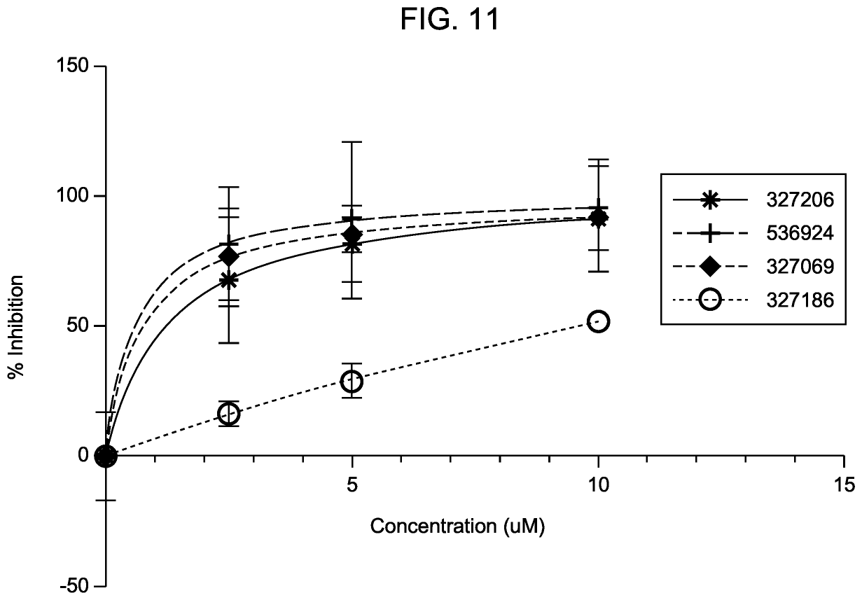

A key aspect of any inhibitor that will be used as a tool to investigate the biological relevancy of hl5-LOX-2 is its efficacy in a cellular model. Currently, there are no potent/specific inhibitors against hl5-LOX-2 which are effective in the cellular milieu. The hl5-LOX-2 inhibitors described herein were tested in HEK293T cells expressing hl5-LOX-2 to inhibit LOX activity and reduce 15-HETE production. The inhibitors, Compound 105, Compound 107 and inhibitor 536924, demonstrated EC₅₀ values of approximately 1 μM (Table 7, FIG. 11), which is consistent with their in vitro IC₅₀ values (Table 2). Compound 106, exhibited weaker potency, with an approximate EC₅₀ of ca. 30 μM. Compound 106 may be less potent due to increased cellular inactivation or decreased cell penetration. Nonetheless, these data demonstrate that the inhibitors described herein can penetrate the cell and inhibit the oxygenation of free, exogenous AA. Additionally, these inhibitors were non-toxic, since cells continued to grow at the same rate as their DMSO controls after treatment with 10 μM of all four inhibitors, confirming them as viable ex vivo tools for examining the activity of hl5-LOX-2. Table 7 summarizes the EC₅₀ values determined in h15-LOX-2/HEK293T cells.

TABLE 7

| Inhibitor | EC₅₀ (μM) | |
|---|---|---|
| Inhibitor 536924 | 0.60 | (0.1) |
| Compound 105 | 0.75 | (0.2) |
| Compound 106 | 31 | (6) |
| Compound 107 | 1.3 | (0.3) |

### CONCLUSIONS

Provided herein is a study of inhibitors of h15-LOX-2, Compound 105, Compound 106 and Compound 107, that are potent and highly selective over other iron dependent oxygenases.

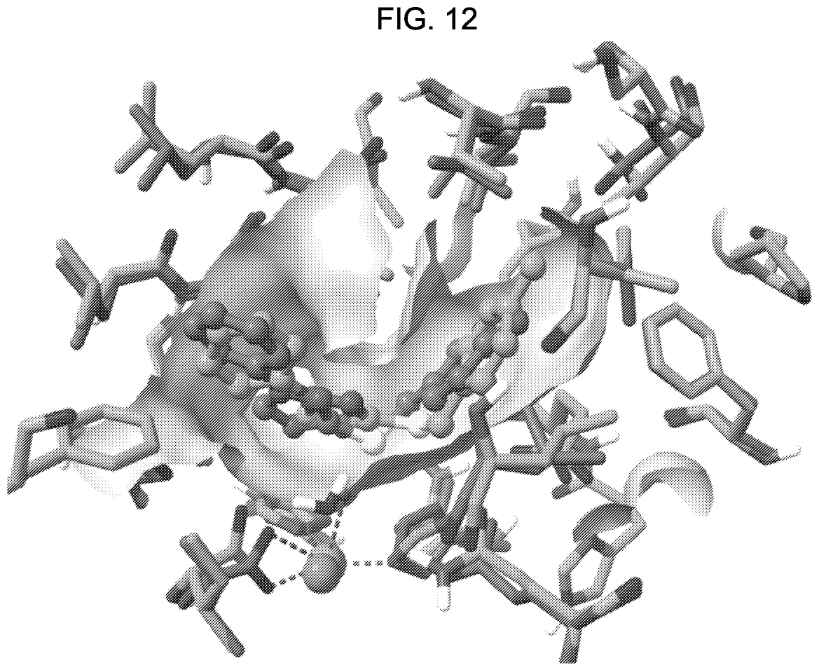

They are mixed-type inhibitors and non-reductive. Each of the compounds perform as potent inhibitors in an HEK293 cell-based assay, indicating their utility as useful chemical tools for biological activity assays and helping to further delineate the role of h15-LOX-2 in certain disease models. In addition, structural and computational data indicate that the inhibitors presented herein bind in the U-shaped channel in similar binding poses (FIG. 12). The HDX results support a similar binding mode between inhibitor 536924 and Compound 105, with the latter restricting protein motion of helix-α2 more robustly, consistent with its greater potency.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a feature in the claim only when the exact phrase “means for” or the exact phrase “step for” is recited at the beginning of such feature in the claim; if such exact phrase is not used in a feature in the claim, then 35 U.S.C. § 112(f) or 35 U.S.C. § 112(6) is not invoked.

What is claimed is:

1. A compound of formula I: 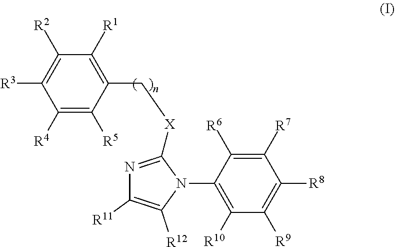
wherein R¹, R², R³, R⁴, R⁵, R⁶, R⁷, R⁸, R⁹ and R¹⁰ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R¹¹ and R¹² are each selected from hydrogen, hydroxy, alkoxy, amine, cyano, thiol, halogen, alkyl, substituted alkyl, haloalkyl, heteroalkyl and substituted heteroalkyl;
X is S or O; and
n is an integer from 0 to 12,
or a salt, solvate or hydrate thereof.

2. The compound according to claim 1, wherein X is S.

3. The compound according to any one of claims 1-2, wherein n is from 1-4.

4. The compound according to any one of claims 1-3, wherein R¹¹ and R¹² are each hydrogen.

5. The compound according to any one of claims 1-4, wherein R¹ is selected from methyl, ethyl, n-propyl, isopropyl, n-butyl, t-butyl, pentyl and hexyl.

6. The compound according to any one of claims 1-5, wherein R³ is a haloalkyl or halogen.

7. The compound according to claim 6, wherein R³ is trifluoromethyl or selected from fluorine, chlorine, bromine and iodine.

8. The compound according to any one of claims 1-5, wherein R³ is an alkyl selected from methyl, ethyl, n-propyl, isopropyl, n-butyl, t-butyl, pentyl and hexyl or thio-substituted C(1-6)alkyl.

9. The compound according to any one of claims 1-8, wherein R² is cyano.

10. The compound according to any one of claims 1-9, wherein the compound is selected from:
1-(p-tolyl)-2-((4-(trifluoromethyl)benzyl)thio)-1H-imidazole (Compound 101): 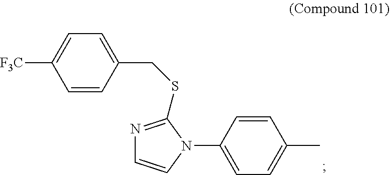 [SMILES: Cc1ccc(-n2ccnc2SCc2ccc(C(F)(F)F)cc2)cc1]
2-((4-fluorobenzyl)thio)-1-phenyl-1H-imidazole (Compound 102): 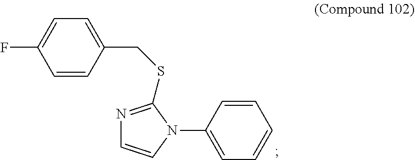 [SMILES: Fc1ccc(CSc2nccn2-c2ccccc2)cc1]
1-(p-tolyl)-2-((4-fluorobenzyl)thio)-1H-imidazole (Compound 102a): 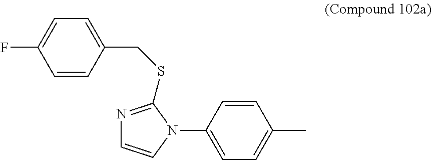 [SMILES: Cc1ccc(-n2ccnc2SCc2ccc(F)cc2)cc1]
2-((4-(methylthio)benzyl)thio)-1-phenyl-1H-imidazole (Compound 103): 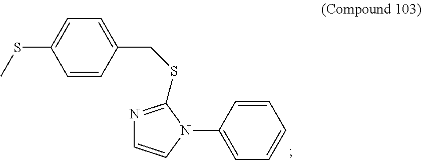 [SMILES: CSc1ccc(CSc2nccn2-c2ccccc2)cc1]
1-(p-tolyl)-2-((4-(methylthio)benzyl)thio)-1H-imidazole (Compound 103a): 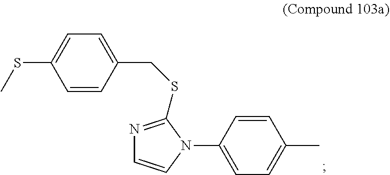
2-fluoro-5-(((1-phenyl-1H-imidazol-2-yl)thio)methyl)benzonitrile (Compound 104): 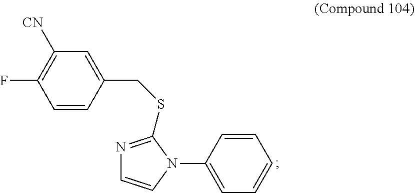
1-(p-tolyl)-2-fluoro-5-(((1H-imidazol-2-yl)thio)methyl)benzonitrile (Compound 104a): 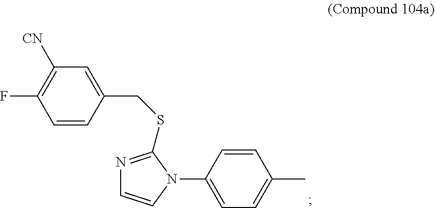
or a salt, solvate or hydrate thereof.

11. A composition comprising:
a compound according to any one of claims 1-10; and
a pharmaceutically acceptable excipient.

12. A method for inhibiting human epithelial 15-(S)-lipoxygenase, the method comprising contacting a cell with a compound according to any one of claims 1-10 or a composition according to claim 11.

13. A method comprising administering to a subject in need thereof a therapeutically effective amount of a compound according to any one of claims 1-10 or a composition according to claim 11.

14. The method according to claim 13, wherein the subject is diagnosed with a cardiovascular disease.

15. The method according to claim 13, wherein the subject is diagnosed with a neurodegenerative disease.
